(19) United States
(12) Patent Application Publication
(10) Pub. No.: US 2022/0305051 A1
(43) Pub. Date: Sep. 29, 2022

(54) NITRITE FORMULATIONS AND USES THEREOF FOR THE TREATMENT OF LUNG INJURY

(71) Applicants: University of Pittsburgh - Of the Commonwealth System of Higher Education, Pittsburgh, PA (US); The UAB Research Foundation, Birmingham, AL (US)

(72) Inventors: Jason Joseph Rose, Fox Chapel, PA (US); Mark Thomas Gladwin, Pittsburgh, PA (US); Rakesh P. Patel, Birmingham, AL (US); Sadis Matalon, Birmingham, AL (US)

(73) Assignees: University of Pittsburgh - Of the Commonwealth System of Higher Education, Pittsburgh, PA (US); The UAB Research Foundation, Birmingham, AL (US)

(21) Appl. No.: 17/640,158

(22) PCT Filed: Sep. 4, 2020
(86) PCT No.: PCT/US2020/049551

§ 371 Date: Mar. 3, 2022

Related U.S. Application Data
(60) Provisional application No. 63/014,902, filed on Apr. 24, 2020.
(60) Provisional application No. 62/896,419, filed on Sep. 5, 2019.

(51) Int. Cl. A61K 33/00; A61P 11/00; A61P 39/00; A61P 31/14; A61K 9/00

(52) U.S. Cl. CPC A61K 33/00; A61P 11/00; A61P 39/00; A61P 31/14; A61K 9/0019; A61K 9/0053; A61K 9/0073

(57) ABSTRACT

Methods for the treatment of lung injury caused by chemical inhalation, smoke inhalation or microbial infection are described. The methods include administering a therapeutically effective amount of nitrite or a nitrite formulation. Nitrite formulations that include a nitrite salt and an anti-caking agent, such as sodium bicarbonate, and the use of such formulations, is also described.

### CROSS REFERENCE TO RELATED APPLICATIONS

This application claims the benefit of U.S. Provisional Application No. 63/014,902, filed Apr. 24, 2020 and U.S. Provisional Application No. 62/896,419, filed Sep. 5, 2019, both of which are herein incorporated by reference in their entirety.

### ACKNOWLEDGMENT OF GOVERNMENT SUPPORT

This invention was made with government support under grant numbers ES023759 and ES02645808 awarded by the National Institutes of Health. The government has certain rights in the invention.

### FIELD

This disclosure concerns formulations of nitrite containing an anti-caking agent, and uses thereof. This disclosure further concerns treatment of lung injuries resulting from chemical or smoke inhalation or viral infection by administration of nitrite.

### BACKGROUND

Exposure to chlorine (Cl₂) gas can cause extensive lung injury, including acute lung injury (ALI) and acute respiratory distress syndrome (ARDS) (White et al., Proc Am Thorac Soc 7:257-263, 2010; Yadav et al., Proc Am Thorac Soc 7:278-283, 2010). Even after cessation of exposure, lung injury can continue to occur for hours or even days, resulting in inflammation, oxidative stress, ALI and ARDS (Samal et al., Proc Am Thorac Soc 7:290-293, 2010; Honavar et al., Am J Respir Cell Mol Biol 45: 419-425, 2010; Martin et al., Am J Respir Crit Care Med 168: 568-574, 2003; Leustik et al., Am J Physiol Lung Cell Mol Physiol 295: L733-L743, 2008; Tuck et al., Respir Res 9:61, 2008). Studies in animal models have demonstrated that administration of antioxidants or β2-agonists following chlorine gas exposure can provide some protection against lung injury (Leustik et al., Am J Physiol Lung Cell Mol Physiol 295: L733-L743, 2008; McGovern et al., Free Radic Biol Med 50:602-608, 2011; McGovern et al., 11: 138, 2010; Zarogiannis et al., Am J Respir Cell Mol Biol 45: 386-392, 2010). Intraperitoneal or intramuscular administration of nitrite has also been shown to reduce ALI in animals exposed to chlorine gas (Samal et al., Free Radic Biol Med 53(7): 1431-1439, 2012; Yadav et al., Am J Physiol Lung Cell Mol Physiol 300(3): L362-369, 2011). However, a needs remains for a safe and effective treatment for inhalational chemical and smoke injuries, as well as lung injury due to viral infection.

### SUMMARY

Treatment of lung injuries resulting from, for example, chemical inhalation, smoke inhalation, microbial infection, trauma or mechanical injury, by administration of nitrite is described. Also described are formulations of nitrite that include a nitrite salt and an anti-caking agent, and use of the formulation for the treatment of a variety of diseases and conditions.

Provided herein is a method of treating a lung injury in a subject by administering to the subject a therapeutically effective amount of a nitrite salt. In some embodiments, the lung injury is caused by inhalation of a chemical, inhalation of smoke, an infection (such as SARS-CoV-2 infection), trauma, or mechanical injury. In particular embodiments, the chemical is not chlorine. In some examples, the nitrite salt is administered as a formulation that includes the nitrite salt and an anti-caking agent, such as sodium bicarbonate.

Also provided is a formulation that includes a nitrite salt and an anti-caking agent, such as sodium bicarbonate. In some embodiments, the anti-caking agent is present at a concentration of at least 20 parts per million. In some embodiments, the nitrite salt is sodium nitrite, potassium nitrite or arginine nitrite.

Further provided are methods of treating a disease or condition in a subject by administering a therapeutically effective amount of the nitrite formulation disclosed herein. In some embodiments, the disease or condition is selected from a lung injury, pulmonary hypertension, heart failure, cardiogenic shock, hypertension, respiratory failure, a metabolic syndrome, diabetes, a lipid disorder, an endocrine disorder, a gastroenterological disorder, hypoperfusion, inflammation, cystic fibrosis and aging.

The foregoing and other objects and features of the disclosure will become more apparent from the following detailed description, which proceeds with reference to the accompanying figures.

### BRIEF DESCRIPTION OF THE DRAWINGS

FIG. 1: Schematic illustrating the effect of Cl₂, Bra or phosgene (COCl₂) gas exposure on NO-scavenging, inflammation, oxidative stress and mitochondrial damage.

FIG. 2: Schematic showing the sources of nitrite as well as the conditions at which nitrite is converted to nitric oxide, an anti-inflammatory agent.

FIGS. 3A-3C: C57bl/6 male mice were anesthetized using ketamine/xylazine and posterior tongues were scraped. Scrapings were incubated in BHI broth for 18 hours at 37° C. to increase bacterial numbers. (FIG. 3A) Nitrate-dependent nitrite formation over time. Nitrite reductase (NR) activity was assessed by adding nitrate and measuring nitrite production. Addition of saline was used as a control. Each line represents an individual mouse sample. (FIG. 3B) Bacterial counts in each sample, as measured by colony forming units (CFU). Each bar represents an individual mouse. (FIG. 3C) Plot of NR activity per CFU. Data are mean±SEM (n=3-5).

FIGS. 4A-4D: Male C57bl/6 mice were exposed to air or Br₂ (600 ppm, 45 minutes). After 24 hours, oral NR activity and bacterial number were determined. (FIG. 4A) Normalized nitrate reductase activity. (FIG. 4B) Bacterial number in colony forming units (CFU). Data are mean±SEM (n=4-5), *P<0.05 by unpaired t-test. Male Sprague Dawley rats were exposed to air (control), Br₂ (FIG. 4C), or C12 (FIG. 4D), at 400 ppm for 30 minutes. At various times thereafter, nitrate-reductase activity was determined by adding nitrate to isolated tongues and measuring nitrite formation rates. Date are mean±SEM (n=3-7), *P<0.05 by 2-way RM-ANOVA with Bonferroni post test (FIG. 4C) or 1-way ANOVA with Tukey post-test (FIG. 4D).

FIG. 5: Plasma nitrite levels 2.5 hours after administration of saline or nitrate (100 mg/kg intraperitoneally) to air-exposed animals. Data are mean±SEM (n=6), *P<0.02 by t-test.

FIGS. 6A-6D: Nitrate-reductase activity (FIG. 6A) and plasma nitrite (FIG. 6B) after administration of chlorohexidine (CHX) or vehicle to the distal tongue in C57bl/6 male mice. *P<0.05 by t-test. (FIG. 6C) Comparison of the most (top 25) abundant microbes on the distal mouse tongue after water or CHX treatment. Each color represents a different bacterial species. Associated bar graphs show bacteria at the family taxa level that were significantly affected by CHX (*p<0.05 or #P<0.07 by t-test, n=4-5). (FIG. 6D) Same as FIG. 6C except for lung microbiome as measured in BAL fluid (*p<0.05 by t-test, n=4-5).

FIGS. 7A-7C: C57bl/6 male mice were treated with water (vehicle) or oral chlorohexidine (CHX) for 7 days, 2× daily and then exposed to C12 (400 ppm, 30 minutes) (FIG. 7A), Br₂ (400 ppm, 30 minutes) (FIG. 7B) or phosgene (10 ppm, 10 minutes) (FIG. 7C) and returned to air. Indicated indices of lung injury were measured in the bronchoalveolar lavage 24 hours after exposure. *P<0.05 relative to air by 1-way ANOVA with Tukey post-test. #P<0.05 relative to Br₂ or COCl₂+ vehicle by 1-way ANOVA with Tukey post-test. All data show mean±SEM (n=3-4).

FIGS. 8A-8C: C57bl/6 male mice were given control or a low nitrate diet for 2 weeks. Plasma nitrate (FIG. 8A) and weight (FIG. 8B) were measured. Then mice were exposed to Cl₂ gas (400 ppm, 30 minutes) and returned to room air. *P<0.05 relative to control diet by unpaired t-test. BAL protein was measured 24 hours thereafter (FIG. 8C). Mice fed a low nitrate diet had considerably higher levels of plasma protein in their BAL at 24 hours post exposure, compared to those that were fed a control diet. #P<0.05 relative to air by 1-way ANOVA with Tukey post-test. *P<0.05 relative to Cl₂ in control diet animals by 1-way ANOVA with Tukey post-test.

FIG. 9A: Male C57bl/6 mice were exposed to Cl₂ gas at 600 ppm for 45 minutes, then brought back to room air and nitrite was administered at 30 minutes or 60 minutes post-exposure by IM injection. Shown are Kaplan-Meier survival curves. Significantly higher survival in mice given nitrite was observed at 60 minutes post-exposure. *P<0.05 between control (saline) and nitrite-treated groups.

FIG. 9B: Male C57bl/6 mice were exposed to Br₂ (400 ppm, 30 minutes) and nitrite (1 mg/kg) was administered at 30 minutes post-exposure by IM injection. Mice were sacrificed at 6 hours or 24 hours post-exposure and BALF cells were measured. Administration of nitrite decreased the number of inflammatory cells in the BAL, an index of lung injury. *P<0.05 by 1-way ANOVA with Tukey post-test relative to Br₂ alone group. Data are mean±SEM (n=4).

FIG. 9C: Male C57bl/6 mice were exposed as in FIG. 9B. At 4 days post-exposure, P. Aeruginosa (10⁵ CFU) was instilled intratracheally, and 24 hours after instillation, bacterial burden in lung homogenates was determined. *P<0.05 relative to air and #P<0.05 relative to Br₂ alone by 1-way ANOVA with Tukey post-test (n=4-5).

FIG. 9D: Male C57bl/6 mice were exposed to Br₂ (600 ppm, 45 minutes) and then 60 minutes after administered IM nitrite (10 mg/kg) or hemopexin (4 μg/g BW), or both, and survival was assessed over 10 days. Shown are Kaplan Meier curves. *p<0.05 relative to saline, n=10.

FIG. 9E: Male C57bl/6 mice were exposed to COCl₂ (10 ppm, 10 minutes) and nitrite (1 mg/kg) was administered at 30 minutes post-exposure by IM injection. Mice were sacrificed at 24 hours post-exposure and BALF protein was measured. Nitrite significantly decreased the concentration of plasma protein in the BAL, the most important index of lung injury. *P<0.05 by 1-way ANOVA with Tukey post-test relative to COCl₂ alone. Data are mean±SEM.

FIGS. 10A-10B: Age-matched male (FIG. 10A) or female (FIG. 10B) 10-week old C57bl/6 mice were exposed to Cl₂ (600 ppm, 45 minutes) and then brought back to room air. Nitrite was administered by IM injection 30 minutes post-exposure and 24 hour survival was assessed. *P<0.05 relative to Cl₂ alone by two-tailed N−1 two proportion test. These data suggest differential therapeutic efficacy for nitrite in males and females; higher doses may be required in females.

### DETAILED DESCRIPTION

Chlorine gas exposure results in increased oxidative stress, inflammation and dysfunction in endogenous repair processes (Bessac and Jordt, Proc Am Thorac Soc 7:269-277, 2010; Chang et al., Toxicol Appl Pharmacol 263:251-258, 2012; Chen et al., Toxicol Appl Pharmacol 272(2): 408-413, 2013; Fanucchi et al., Am J Respir Cell Mol Biol 46:599-606, 2012; Gessner et al., Am J Physiol Lung Cell Mol Physiol 304:L765-773, 2013; Honavar et al., Am J Respir Cell Mol Biol 45(2): 419-425, 2010; Koohsari et al., Respir Res 8:21, 2007; Leustik et al., Am J Physiol Lung Cell Mol Physiol 295:L733-743, 2008; Martin et al., Am J Respir Crit Care Med 168:568-574, 2003; McGovern et al., Free Radic Biol Med 50:602-608, 2011; McGovern et al., Respir Res 11:138, 2010; Musah et al., Respir Res 13:107, 2012; O'Koren et al., Am J Respir Cell Mol Biol 49(5): 788-797, 2013; Song et al., Am J Respir Cell Mol Biol 45:88-94, 2011; Yadav et al., Am J Physiol Lung Cell Mol Physiol 300:L362-369, 2011; Zarogiannis et al., Am J Respir Cell Mol Biol 45:386-392, 2011). The endothelial dysfunction associated with chlorine injury is characterized by a loss of nitric oxide (NO) bioavailability, which predisposes tissues to inflammation and oxidative stress. Repleting NO reduces inflammation and oxidative stress and improves survival. Available data also suggests that antioxidant (vitamin C) and chelator (desferoxamine) therapies can improve survival and enhance lung epithelial repair (Fanucchi et al., Am J Respir Cell Mol Biol 46:599-606, 2012).

The present disclosure describes the administration of nitrite in intramuscular, intravenous, subcutaneous, oral or inhalational forms to treat post-exposure chemical lung injury due to chemical inhalation or smoke inhalation, as well lung injury resulting from infectious disease, such as SARS-CoV-2. Administration of nitrite as soon as possible post exposure to chemicals, smoke or infectious agents is proposed to improve lung injury and survival.

As demonstrated in the Examples herein, post-exposure treatment with intramuscular sodium nitrite resulted in significant decreases in acute lung injury and improved survival in mice, rats and rabbits after exposure to chlorine, bromine and/or phosgene gas (see also Honavar et al., Toxicol Lett 271:20-25, 2017; Honavar et al., Am J Physiol Lung Cell Mol Physiol 307(11):L888-L894, 2014; Samal et al., Free Radic Biol Med 53(7):1431-1439, 2012).

### I. Abbreviations

ALI acute lung injury

ARDS acute respiratory distress syndrome

BAL bronchoalveolar lavage

BALF bronchoalveolar lavage fluid

Br₂ bromine

CFU colony forming unit

CHX chlorohexidine

Cl₂ chlorine

COCl₂ phosgene

CoV coronavirus

ELISA enzyme-linked immunosorbent assay

eNOS endothelial nitric oxide synthase

GI gastrointestinal

Hb hemoglobin

IM intramuscular

IP intraperitoneal

NO nitric oxide

NOS nitric oxide synthase

NR nitrite reductase

PMN polymorphonuclear

ppm parts per million

RBC red blood cell

SARS severe acute respiratory syndrome

### II. Terms and Methods

Unless otherwise noted, technical terms are used according to conventional usage. Definitions of common terms in molecular biology may be found in Benjamin Lewin, Genes X, published by Jones & Bartlett Publishers, 2009; and Meyers et al. (eds.), The Encyclopedia of Cell Biology and Molecular Medicine, published by Wiley-VCH in 16 volumes, 2008; and other similar references.

As used herein, the singular forms “a,” “an,” and “the,” refer to both the singular as well as plural, unless the context clearly indicates otherwise. For example, the term “a salivary gland” includes single or plural cells and can be considered equivalent to the phrase “at least one salivary gland.” As used herein, the term “comprises” means “includes.” It is further to be understood that any and all base sizes or amino acid sizes, and all molecular weight or molecular mass values, given for nucleic acids or polypeptides are approximate, and are provided for descriptive purposes, unless otherwise indicated. Although many methods and materials similar or equivalent to those described herein can be used, particular suitable methods and materials are described herein. In case of conflict, the present specification, including explanations of terms, will control. In addition, the materials, methods, and examples are illustrative only and not intended to be limiting. To facilitate review of the various embodiments, the following explanations of terms are provided:

Administration: To provide or give a subject an agent, such as nitrite, by any effective route. Exemplary routes of administration include, but are not limited to, injection (such as subcutaneous, intramuscular, intradermal, intraperitoneal, intravenous, and intratumoral), intraglandular, oral, sublingual, rectal, transdermal, intranasal, vaginal and inhalation routes.

Anti-caking agent: An agent that inhibits or prevents caking of a drug substance, such as a nitrite salt. In the context the present disclosure, the formulation of nitrite salt generally contains an anti-caking agent in at least 20 parts per million. Examples of anti-caking agents include, but are not limited to, sodium bicarbonate, aluminum caprate, aluminum caprylate, aluminum laurate, aluminum myristate, aluminum oleate, aluminum palmitate, aluminum salts of fatty acids, aluminum stearate, calcium caprate, calcium caprylate, calcium carbonate, calcium chloride, calcium myristate, calcium oxide, calcium palmitate, calcium phosphate (dibasic or tribasic), calcium salts of fatty acids, calcium silicate, calcium stearate, calcium sulfate, carboxymethyl cellulose, carnauba wax, carrageenan, castor oil, microcrystalline cellulose, dextrin, dextrose, glycerin, glycerin monooleate, iron ammonium citrate, magnesium caprate, magnesium caprylate, magnesium carbonate, magnesium laurate, magnesium myristate, magnesium oleate, magnesium oxide, magnesium palmitate, magnesium salts of fatty acids, magnesium silicate, magnesium stearate, magnesium sulfate, maltodextrin, mannitol, potassium caprate, potassium caprylate, potassium laurate, potassium myristate, potassium palmitate, potassium permanganate, potassium salts of fatty acids, propylene glycol, silicon dioxide, sodium aluminum phosphate (acidic or basic), sodium aluminum silicate, sodium caprate, sodium caseinate, sodium chloride, sodium laurate, sodium mono- and dimethyl naphthalene sulfonates, sodium myristate, sodium oleate, sodium palmitate, sodium phosphate (monobasic, dibasic or tribasic), sodium pyrophosphate, sodium salts of fatty acids, sodium silicate, sodium stearate, sodium tripolyphosphate, tartaric acid, titanium dioxide, and xanthan gum.

Bromine (Br₂): A naturally occurring element that is a liquid at room temperature. Bromine has a dark reddish-brown color with a pungent bleach-like odor. Bromine gas is toxic by inhalation, causing damage to mucous membranes and other tissues, such as the lung. Bromine liquid or gas can cause skin irritation and burns. Inhalation of bromine gas can cause long-term injury to the lungs, as well as kidney and brain damage.

Chemical lung injury: Lung injury as a result of exposure to a chemical, such as bromide, methyl bromide, mustard gas (sulfur mustard), nitrogen mustard (HN-1, HN-2, HN-3), phosgene, phosgene oxime, diphosgene, phosphine, ammonia, bromine, methyl isocyanate, hydrogen chloride, chlorine, osmium tetroxide, phosphorous (elemental, white or yellow), sulfuryl fluoride, lewisite, riot control agents (such as chloroacetophenone (CN), chlorobenzylidenemalononitrile, chloropicrin, bromobenzylcyanide (CA) and/or dibenzoxazepine (CR)).

Heart failure: A condition that occurs when the heart is no longer capable of pumping enough blood to other parts of the body. Types of heart failure include left-sided heart failure, right-sided heart failure and congestive heart failure. Left-sided heart failure is the most common type and includes heart failure with reduced ejection fraction (HFrEF) (also known as systolic failure) and heart failure with preserved ejection fraction (HFpEF) (also known as diastolic failure). With HFrEF, the left ventricles lose their ability to contract normally and the heart can't pump with enough force to circulate blood. Blood then builds up in the pulmonary veins, which causes shortness of breath. In subjects with HFpEF, the left ventricles lose their ability to relax, and the heart can't properly fill with blood between each heartbeat. Right-sided heart failure (also known as right ventricular heart failure) typically occurs as a result of left-sided heart failure. With right-sided heart failure, the heart is too weak to pump enough blood to the lungs so blood builds up in the veins. With congestive heart failure (CHF), as blood flow out of the heart slows, blood returning to the heart backs up, resulting in congestion in body tissues. CHF often results in swelling of the legs, ankles and other parts of the body.

Lung injury: In the context of the present disclosure, “lung injury” encompasses any disease, disorder, condition or injury that causes damage to the lung. Examples of lung injuries include, but are not limited to, inhalation chemical injuries, inhalation smoke injuries, injuries resulting from viral, bacterial or fungal infection, trauma or mechanical injuries. Lung injuries can result in acute respiratory distress syndrome or acute lung injury from any disease, disorder or condition, including sepsis, trauma, bacterial infection, viral infection, fungal infection and drug reaction. In some instances, lung injury is caused by pneumonia. In specific examples, the lung injury is caused by a viral infection, such as SARS-CoV-2 infection.

Nitrite: The inorganic anion⁻NO₂ or a salt of nitrous acid (NO₂⁻). Nitrites are often highly soluble, and can be oxidized to form nitrates or reduced to form nitric oxide or ammonia. Nitrite may form salts with alkali metals, such as sodium (NaNO₂, also known as nitrous acid sodium salt), potassium and lithium, with alkali earth metals, such as calcium, magnesium and barium, with organic bases, such as amine bases, for example, dicyclohexylamine, pyridine, arginine, lysine and the like. Other nitrite salts may be formed from a variety of organic and inorganic bases. In particular embodiments, the nitrite is a salt of an anionic nitrite delivered with a cation, which cation is selected from sodium, potassium, and arginine. Many nitrite salts are commercially available, and/or readily produced using conventional techniques.

Parenteral: Administered outside of the intestine, for example, not via the alimentary tract. Generally, parenteral formulations are those that will be administered through any possible mode except ingestion. This term especially refers to injections, whether administered intravenously, intrathecally, intramuscularly, intraperitoneally, or subcutaneously, and various surface applications including intranasal, intradermal, and topical application, for instance.

Pharmaceutically acceptable carrier: The pharmaceutically acceptable carriers useful in this disclosure are conventional. Remington: The Science and Practice of Pharmacy, The University of the Sciences in Philadelphia, Editor, Lippincott, Williams, & Wilkins, Philadelphia, Pa., 21^{st} Edition (2005), describes compositions and formulations Suitable for pharmaceutical delivery of the compounds herein disclosed. In general, the nature of the carrier will depend on the particular mode of administration being employed. For instance, parenteral formulations usually comprise injectable fluids that include pharmaceutically and physiologically acceptable fluids such as water, physiological saline, balanced salt solutions, aqueous dextrose, glycerol or the like as a vehicle. For solid compositions (for example, powder, pill, tablet, or capsule forms), conventional non-toxic solid carriers can include, for example, pharmaceutical grades of mannitol, lactose, starch, or magnesium stearate. In addition to biologically-neutral carriers, pharmaceutical compositions to be administered can contain minor amounts of non-toxic auxiliary substances, such as wetting or emulsifying agents, preservatives, and pH buffering agents and the like, for example sodium acetate or sorbitan monolaurate.

Phosgene (carbonyl chloride, CoCl₂): An industrial chemical used to make plastics and pesticides. At room temperature, phosgene is a gas. Phosgene gas can appear colorless or white to pale yellow, and has an odor of newly mown hay at low concentrations, and a stronger unpleasant odor at higher concentrations. Exposure to phosgene gas can result in damage to the skin, eyes, nose, throat and lungs. Signs of exposure include coughing, burning in the eyes and throat, watery eyes, blurred vision, difficulty breathing, nausea and vomiting. Delayed effects of phosgene gas exposure include low blood pressure, pulmonary edema and heart failure.

Preventing or treating a disease: “Preventing” a disease refers to inhibiting the full development of a disease. “Treatment” refers to a therapeutic intervention that ameliorates a sign or symptom of a disease or pathological condition after it has begun to develop.

Pulmonary hypertension: Abnormally elevated blood pressure in the pulmonary circulation. Pulmonary hypertension affects the arteries in the lungs and right side of the heart. In this condition, the pulmonary arteries can become stiff, swollen and thick, which blocks or slows blood flow, leading to pulmonary hypertension.

Purified: The term purified does not require absolute purity; rather, it is intended as a relative term. Thus, for example, a purified nitrite salt preparation is one in which the specified nitrite salt is more enriched than it is in its generative environment, for instance within a biochemical reaction chamber. Preferably, a preparation of a specified nitrite salt is purified such that the salt represents at least 50% of the total nitrite content of the preparation. In some embodiments, a purified preparation contains at least 60%, at least 70%, at least 80%, at least 85%, at least 90%, at least 95% or more of the specified compound, such as a particular nitrite salt.

Pneumonia: An infection in one or both lungs caused by bacteria, viruses or fungi. Pneumonia causes inflammation of the alveoli of the lung, resulting in accumulation of fluid in the alveoli, which can cause difficulty in breathing.

Respiratory failure: A condition that occurs when not enough oxygen passes from the lungs into the blood. Respiratory failure can be either acute or chronic. Diseases and disorders that impair breathing can lead to respiratory failure. Examples includes chronic obstructive lung disease (COPD), pneumonia, acute respiratory syndrome, pulmonary embolism, cystic fibrosis, and acute lung injuries, such as inhalation of harmful chemicals or smoke.

Severe acute respiratory syndrome coronavirus 2 (SARS-CoV-2): A betacoronavirus that causes coronavirus disease referred to as COVID-19 (coronavirus disease 2019). SARS-CoV-2 was first reported in Wuhan, China in late December 2019. Infection with SARS-CoV-2 can cause fever, chills, cough, pneumonia, difficulty breathing, respiratory failure, heart failure and in some cases, death.

Standard methods for detecting viral infection may be used to detect SARS-CoV-2 infection, including but not limited to, assessment of patient symptoms and background and genetic tests such as reverse transcription-polymerase chain reaction (rRT-PCR). The test can be done on patient samples such as respiratory or blood samples.

Sodium bicarbonate: A chemical compound with the formula NaHCO₃. Sodium bicarbonate is also known as sodium hydrogen carbonate, baking soda and bicarbonate of soda. Sodium bicarbonate can be used as an anti-caking agent.

Subject: Living multi-cellular vertebrate organisms, a category that includes both human and veterinary subjects, including human and non-human mammals.

Therapeutically effective amount: A quantity of a compound, such as a nitrite salt, sufficient to achieve a desired effect in a subject being treated. For instance, this can be the amount necessary to treat or ameliorate a lung injury, or to measurably decrease hypertension in a subject.

An effective amount of a compound, such as a nitrite salt, can be administered in a single dose, or in several doses, for example daily, during a course of treatment. However, the effective amount will be dependent on the particular compound applied, the subject being treated, the severity and type of the affliction, and the manner of administration of the compound. For example, a therapeutically effective amount of an active ingredient can be measured as the concentration (moles per liter or molar-M) of the active ingredient (such as a pharmaceutically-acceptable salt of nitrite) in blood (in vivo) or a buffer (in vitro) that produces an effect. Alternatively, the therapeutically effective amount can be measured by weight, such as in milligrams, grams or kilograms.

In some embodiments, the therapeutically effective amount is an amount sufficient to achieve about 0.5 to about 500 μM final concentration of nitrite in the circulating blood of a subject, which level can be determined empirically or through calculations. In specific examples, the concentration of nitrite in the circulation is about 1 to about 250 μM, about 2.5 to about 200 μM, about 5 to about 100 μM, or about 10 to about 50 μM. In other embodiments, the therapeutically effective amount of a nitrite salt is less than about 300 mg or less nitrite in a single dose, or a dose provided over a period of time (e.g., by infusion or inhalation). In some examples, the effective amount is about 10, about 20, about 30, about 40, about 50, about 60, about 70, about 80, about 90, about 100, about 150, about 200, about 250 or about 300 mg. Specific example dosages of nitrite salts are provided herein, though the examples are not intended to be limiting. Exact dosage amounts will vary by the size of the subject being treated, the duration of the treatment, the mode of administration, and so forth.

Particularly beneficial therapeutically effective amounts of a nitrite salt (e.g., sodium nitrite), are those that are effective for treating lung injury (or another disease or condition), but not so high that a significant or toxic level of methemoglobin is produced in the subject to which the nitrite salt is administered. In specific embodiments, for instance, no more than about 25% methemoglobin is produced in the subject. In specific examples, no more than 20%, no more than 15%, no more than 10%, no more than 8% or less methemoglobin is produced, for instance as little as 5% or 3% or less, in response to treatment with the nitrite salt.

### III. Overview of Embodiments

It is disclosed herein that in animal models of chlorine, bromine and/or phosgene gas poisoning, post-exposure treatment with nitrite results in significant decreases in acute lung injury and improved survival. In view of these findings, the present disclosure describes the administration of nitrite in intramuscular, intravenous, subcutaneous, oral or inhalational forms to treat post-exposure chemical lung injury due to chemical inhalation or smoke inhalation, as well lung injury resulting from an infectious disease, such as viral infection, for example SARS-CoV-2. Administration of nitrite as soon as possible post exposure to chemicals, smoke or infectious agents is proposed to improve lung injury and survival.

Provided herein are methods for the treatment of lung injury caused by chemical inhalation, smoke inhalation, viral infection, bacterial infection or fungal infection. In some embodiments, the methods include administering a therapeutically effective amount of nitrite, such as a nitrite salt, or a formulation thereof. In some examples, the chemical causing the lung injury is not chlorine (Cl₂).

In some embodiments, the lung injury is caused by a chemical, and the chemical includes bromide, methyl bromide, mustard gas, nitrogen mustard, phosgene, phosgene oxime, diphosgene, phosphine, ammonia, bromine, methyl isocyanate, hydrogen chloride, osmium tetroxide, phosphorous, sulfuryl fluoride, lewisite, chloroacetophenone, chlorobenzylidenemalononitrile, chloropicrin, bromobenzylcyanide, dibenzoxazepine, or a combination or two or more, three or more or four or more thereof. In some examples, the chemical comprises bromine or phosgene gas.

In some embodiments, the lung injury is caused by smoke, and the smoke is from a fire, exhaust fumes or an explosion.

In other embodiments, the lung injury is caused by a viral infection, such as an infection by a coronavirus, for example SARS-CoV-2. In some examples, the subject being treated has pneumonia or respiratory failure resulting from SARS-CoV-2 infection. The lung injury associated with SARS-CoV-2 can cause pneumonia alterations of pulmonary hemodynamics, shock and hypoxia.

In some embodiments, the nitrite salt is sodium nitrite, potassium nitrite or arginine nitrite.

In some embodiments, the therapeutically effective amount of nitrite salt is about 5 mg to about 600 mg, such as about 10 mg to about 300 mg, about 20 mg to about 200 mg, about 30 mg to about 150 mg, about 40 mg to about 100 mg or about 50 to about 75 mg of nitrite salt. In some examples, the therapeutically effective amount is 600 mg or less, 500 mg or less, 400 mg or less, 300 mg or less, 275 mg or less, 250 mg or less, 225 mg or less, 200 mg or less, 175 mg or less, 150 mg or less, 125 mg or less, 100 mg or less, 75 mg or less, or 50 mg or less of nitrite salt.

In some examples, the therapeutically effective amount of nitrite salt is about 0.5 to about 10 mg/kg, such as about 2 to about 8 mg/kg, about 2 to about 6 mg/kg or about to about 8 mg/kg. In specific examples, the therapeutically effective amount of nitrite salt is about 1 mg/kg, about 2 mg/kg, about 3 mg/kg, about 4 mg/kg, about 5 mg/kg, about 6 mg/kg, about 7 mg/kg, about 8 mg/kg, about 9 mg/kb or about 10 mg/kg. In specific non-limiting embodiments, the therapeutically effective amount of nitrite salt does not exceed 150 mg, 300 mg, 450 mg or 600 mg.

In some embodiments, the nitrite salt is administered as a formulation that includes the nitrite salt and an anti-caking agent. In some examples, the anti-caking agent is selected from any one of sodium bicarbonate, aluminum caprate, aluminum caprylate, aluminum laurate, aluminum myristate, aluminum oleate, aluminum palmitate, aluminum salts of fatty acids, aluminum stearate, calcium caprate, calcium caprylate, calcium carbonate, calcium chloride, calcium myristate, calcium oxide, calcium palmitate, calcium phosphate (dibasic or tribasic), calcium salts of fatty acids, calcium silicate, calcium stearate, calcium sulfate, carboxymethyl cellulose, carnauba wax, carrageenan, castor oil, microcrystalline cellulose, dextrin, dextrose, glycerin, glycerin monooleate, iron ammonium citrate, magnesium caprate, magnesium caprylate, magnesium carbonate, magnesium laurate, magnesium myristate, magnesium oleate, magnesium oxide, magnesium palmitate, magnesium salts of fatty acids, magnesium silicate, magnesium stearate, magnesium sulfate, maltodextrin, mannitol, potassium caprate, potassium caprylate, potassium laurate, potassium myristate, potassium palmitate, potassium permanganate, potassium salts of fatty acids, propylene glycol, silicon dioxide, sodium aluminum phosphate (acidic or basic), sodium aluminum silicate, sodium caprate, sodium caseinate, sodium chloride, sodium laurate, sodium mono- and dimethyl naphthalene sulfonates, sodium myristate, sodium oleate, sodium palmitate, sodium phosphate (monobasic, dibasic or tribasic), sodium pyrophosphate, sodium salts of fatty acids, sodium silicate, sodium stearate, sodium tripolyphosphate, tartaric acid, titanium dioxide, and xanthan gum. In specific examples, the anti-caking agent is sodium bicarbonate.

In some examples, the concentration of the anti-caking agent in the nitrite formulation is at least 20 parts per million (ppm), such as at least 25 ppm, at least 30 ppm, at least 35 ppm, at least 40 ppm, at least 45 ppm, or at least 50 ppm. In specific non-limiting examples, the anti-caking agent is sodium bicarbonate and the nitrite formulation includes at least 20 ppm of sodium bicarbonate.

In some embodiments, the nitrite salt is administered by an intramuscular, intravenous, subcutaneous, oral or inhalation route.

In some embodiments, the method further includes selecting a subject with a lung injury prior to administering the nitrite salt.

Also provided herein are formulations of nitrite. In some embodiments, the formulation includes a nitrite salt and an anti-caking agent. In some examples, the anti-caking agent is present at a concentration of at least 20 parts per million, such as at least 25 ppm, at least 30 ppm, at least 35 ppm, at least 40 ppm, at least 45 ppm, or at least 50 ppm.

In some embodiments of the nitrite formulation, the anti-caking agent is selected from any one of sodium bicarbonate, aluminum caprate, aluminum caprylate, aluminum laurate, aluminum myristate, aluminum oleate, aluminum palmitate, aluminum salts of fatty acids, aluminum stearate, calcium caprate, calcium caprylate, calcium carbonate, calcium chloride, calcium myristate, calcium oxide, calcium palmitate, calcium phosphate (dibasic or tribasic), calcium salts of fatty acids, calcium silicate, calcium stearate, calcium sulfate, carboxymethyl cellulose, carnauba wax, carrageenan, castor oil, microcrystalline cellulose, dextrin, dextrose, glycerin, glycerin monooleate, iron ammonium citrate, magnesium caprate, magnesium caprylate, magnesium carbonate, magnesium laurate, magnesium myristate, magnesium oleate, magnesium oxide, magnesium palmitate, magnesium salts of fatty acids, magnesium silicate, magnesium stearate, magnesium sulfate, maltodextrin, mannitol, potassium caprate, potassium caprylate, potassium laurate, potassium myristate, potassium palmitate, potassium permanganate, potassium salts of fatty acids, propylene glycol, silicon dioxide, sodium aluminum phosphate (acidic or basic), sodium aluminum silicate, sodium caprate, sodium caseinate, sodium chloride, sodium laurate, sodium mono- and dimethyl naphthalene sulfonates, sodium myristate, sodium oleate, sodium palmitate, sodium phosphate (monobasic, dibasic or tribasic), sodium pyrophosphate, sodium salts of fatty acids, sodium silicate, sodium stearate, sodium tripolyphosphate, tartaric acid, titanium dioxide, and xanthan gum. In specific examples, the anti-caking agent is sodium bicarbonate. In specific non-limiting examples, the anti-caking agent is sodium bicarbonate and the nitrite formulation includes at least 20 ppm of sodium bicarbonate.

In some embodiments, the formulation includes a phosphate buffered solution, a lactated Ringer's solution or physiological saline. In some examples, the phosphate buffered solution, lactated Ringer's solution or physiological saline is sterile. In some examples, the phosphate buffer is comprised of sodium hydroxide, phosphoric acid, sodium phosphate (monobasic; dibasic or a combination thereof), or any combination thereof.

In some embodiments, the formulation includes water.

In some embodiments, the nitrite salt of the formulation is sodium nitrite, potassium nitrite or arginine nitrite.

In some embodiments, the concentration of nitrite salt in the formulation is about 30 mg/ml, about 40 mg/ml, about 60 mg/ml or about 80 mg/ml. In some embodiments, the formulation is provided in unit dose form. In some examples, such as when the formulation is administered intramuscularly, the total volume of the unit dose is about 0.5 ml, about 1 ml, about 3 ml, about 5 ml, about 8 ml, about 10 ml or about 20 ml.

Further provided are methods of treating a disease or condition in a subject by administering to the subject a therapeutically effective amount of a nitrite formulation disclosed herein. In some embodiments, the disease or condition is selected from a lung injury, pulmonary hypertension, heart failure, cardiogenic shock, hypertension, respiratory failure, a metabolic syndrome, diabetes, a lipid disorder, an endocrine disorder, a gastroenterological disorder, hypoperfusion, inflammation, cystic fibrosis and aging.

In some embodiments, the disease or condition is a lung injury and the lung injury is caused by inhalation of a chemical, inhalation of smoke, SARS-CoV-2 infection, trauma or mechanical injury. In some examples, the lung injury is caused by a chemical, and the chemical includes bromide, methyl bromide, mustard gas, nitrogen mustard, phosgene, phosgene oxime, diphosgene, phosphine, ammonia, bromine, methyl isocyanate, hydrogen chloride, osmium tetroxide, phosphorous, sulfuryl fluoride, lewisite, chloroacetophenone, chlorobenzylidenemalononitrile, chloropicrin, bromobenzylcyanide, dibenzoxazepine, or a combination or two or more, three or more or four or more thereof. In some examples, the chemical comprises bromine or phosgene gas.

In some examples, the lung injury is caused by smoke, and the smoke is from a fire, exhaust fumes or an explosion. In other examples, the lung injury is caused by a viral infection, such as an infection by SARS-CoV-2. In particular non-limiting examples, the subject being treated has pneumonia or respiratory failure resulting from SARS-CoV-2 infection.

In some embodiments of the method, the disease or condition comprises pulmonary hypertension and heart failure with a preserved ejection fraction (HFpEF).

In other embodiments, the disease or condition is heart failure, and the heart failure includes right-sided heart failure, left-sided heart failure or congestive heart failure.

In other embodiments, the disease or condition is hypertension, and the hypertension includes chronic hypertension, acute hypertension, urgency hypertension, emergency hypertension, prehypertension, or a combination thereof.

In other embodiments, the disease or condition is respiratory failure and the respiratory failure comprises acute respiratory distress syndrome, respiratory failure from trauma or mechanical injury, respiratory failure due to pneumonia, respiratory failure due to trauma, respiratory failure due to lung transplantation, respiratory failure due to lung transplantation rejection (acute and chronic), respiratory failure resulting from an infectious disease, respiratory failure resulting from pulmonary edema, respiratory failure resulting from pulmonary embolism, respiratory failure due to infant respiratory distress syndrome (IRDS; also known as neonatal respiratory distress syndrome (NRDS), respiratory distress syndrome of newborn, persistent pulmonary hypertension of the newborn (PPHN) and surfactant deficiency disorder (SDD), respiratory failure due to interstitial lung disease, or respiratory failure resulting from an autoimmune disease.

In some embodiments, the disease or condition is a lipid disorder and the lipid disorder includes hypercholesterolemia, hypertriglyceridemia, or both.

In some embodiments, the method further includes selecting a subject with a lung injury, pulmonary hypertension, heart failure, cardiogenic shock, hypertension, respiratory failure, a metabolic syndrome, diabetes, a lipid disorder, an endocrine disorder, a gastroenterological disorder, hypoperfusion, inflammation, cystic fibrosis or aging prior to administering the formulation.

Also provided herein is a method of enhancing cardiovascular performance in a subject by administering to the subject a therapeutically effective amount of the nitrite formulation disclosed herein.

Further provided is a method of providing a nutritional supplement to a subject, by administering to the subject the nitrite formulation disclosed herein.

The present disclosure further contemplates intramuscular, intravenous, subcutaneous, oral or inhalational administration of the disclosed nitrite formulations during the perioperative period. Also contemplated are use of the disclosed nitrite formulations as an antidote (such as an antidote for cyanide poisoning); use of the disclosed nitrite formulations as dietary supplements; use of the disclosed nitrite formulations to enhance cardiovascular performance; use of the disclosed nitrite formulations to improve oxygen delivery; use of the disclosed nitrite formulations to treat microbial infections and/or to inhibit microbial (such as bacterial) activity; and use of the disclosed nitrite formulations for alteration of the microbiome.

In some embodiments, the subject is administered an effective amount of the nitrite formulation, wherein the effective amount contains about 5 mg to about 600 mg, such as about 10 mg to about 300 mg, about 20 mg to about 200 mg, about 30 mg to about 150 mg, about 40 mg to about 100 mg or about 50 to about 75 mg of nitrite salt. In some examples, the effective amount contains 600 mg or less, 500 mg or less, 400 mg or less, 300 mg or less, 275 mg or less, 250 mg or less, 225 mg or less, 200 mg or less, 175 mg or less, 150 mg or less, 125 mg or less, 100 mg or less, 75 mg or less, or 50 mg or less of nitrite salt. In some examples, the effective amount contains about 0.5 to about 10 mg/kg, such as about 2 to about 8 mg/kg, about 2 to about 6 mg/kg or about to about 8 mg/kg of nitrite salt. In specific examples, the effective amount contains about 1 mg/kg, about 2 mg/kg, about 3 mg/kg, about 4 mg/kg, about 5 mg/kg, about 6 mg/kg, about 7 mg/kg, about 8 mg/kg, about 9 mg/kb or about 10 mg/kg of nitrite salt. In specific non-limiting embodiments, the effective amount does not exceed 150 mg, 300 mg, 450 mg or 600 mg of nitrite salt.

In some embodiments, the concentration of nitrite salt in the nitrite formulation is about 30 mg/ml, about 40 mg/ml, about 60 mg/ml or about 80 mg/ml. In some embodiments, the formulation is provided in unit dose form. In some examples, such as when the formulation is administered intramuscularly, the total volume of the unit dose is about 0.5 ml, about 1 ml, about 3 ml, about 5 ml, about 8 ml, about 10 ml or about 20 ml.

### IV. Inhalational Chemical and Smoke Injuries

Common features of injury caused by exposure to reactive gases include acute injury to point of contact areas (airways, skin) that is followed by acute and chronic inflammatory injury to the cardiopulmonary system. Exposure to chlorine (Cl₂), bromine (Br₂), or phosgene (COCl₂) forms intermediates that cause red blood cell (RBC) hemolysis (Aggarwal et al., Toxicol Lett 312: 204-213, 2019; Aggarwal et al., JCI Insight 3(21), doi: 10.1172/jci.insight.120694, 2018; Aggarwal et al., Antioxid Redox Signal 24(2): 99-112, 2015). Released free hemoglobin and heme collectively decrease nitric oxide (NO) bioavailability, and promote mitochondrial damage, inflammation and oxidative stress. NO is an endogenous anti-inflammatory agent. While NO-production is regulated by NO-synthase enzymes, data indicate non-NO synthase sources of NO are also significant in mammals, specifically from dietary nitrate present in “healthy diets” comprising green leafy and root vegetables. Upon ingestion, nitrate is concentrated 10-20 fold into the saliva, where it is reduced to nitrite by commensal lingual nitrate-reductase (NR) expressing bacteria. Nitrite is swallowed, increases in the plasma and then mediates NO-signaling in all major tissues by a further 1-electron reduction process. Thus, it is believed that oral nitrate-reducing bacteria are significant mediators of physiologic mammalian NO-homeostasis.

A. Halogen and Phosgene Toxicity Mechanisms

Chlorine (Cl₂) and bromine (Br₂) are halogens used in various industrial processes and consequently stored and transported in large amounts. Phosgene (COCl₂) is a high irritant gas currently used in the production of dyes, pesticides, and plastics, and is a breakdown product of chloroform. Exposure to COCl₂ causes lung injury and inflammation (Aggarwal et al., Toxicol Lett 312: 204-213, 2019).⁸⁻¹⁰ Cl₂, Br₂ and COCl₂ are all inhaled chemical threat agents. Accidental exposure in these settings, as well as intentional exposure in the military arena, have been documented, with facilities producing these gases considered by the Department of Homeland Security at high risk for terrorist attacks. The acute and chronic health effects of Cl₂, Br₂ or COCl₂ exposure remain under investigation. During exposure to these gases, injury to the airways occurs and is then followed, over hours to days, by further airway/alveolar epithelial injury and pulmonary and systemic endothelial injury.¹¹⁻¹³ Collectively, this manifests as progressively worsening acute lung injury (ALI), development of reactive airways, pulmonary and systemic vascular dysfunction (blood flow) and impaired cardiac contractility. While chemically distinct, the post-exposure injury phenotype is similar for all three gases. Moreover, the molecular and biochemical mechanisms underlying injury are believed to be similar. The present disclosure describes the finding that Cl₂, Br₂ or COCl₂ exposure results in hemolysis; the released hemoglobin/heme then promotes post-exposure toxicity by promoting oxidative stress and inflammation in the lung, inhibiting nitric oxide (NO) bioavailability and causing mitochondrial DNA damage and cellular bioenergetic dysfunction.

B. NO-Homeostasis Mechanisms: Role of Diet and the Oral Microbiome

One contributing mechanism to post-Cl₂ induced toxicity is dysregulation of endogenous nitric oxide (NO)-formation^{6,12}, but mechanisms remain to be elucidated. Nitric oxide mediates vasodilation, limits inflammation and coagulation, and regulates cell death, proliferation and mitochondrial function^{14,15}. Consequently, inhibition of this pathway predisposes to and/or leads to numerous acute and chronic inflammatory diseases. While NO-formation from nitric oxide synthases is well-accepted, it is now evident that mammalian NO-signaling is also regulated by non-enzymatic sources of NO. Typically, NO is thought to be inactivated by its oxidation to nitrite and nitrate. However, nitrite is not inert, and can be reduced (by 1-electron) back to NO or other NO-containing intermediates (e.g. S-nitrosothiols) during inflammation and hypoxia¹⁶⁻²⁰. In this model, endogenous or therapeutic nitrite is a source of NO-signaling equivalents, especially during stress when endogenous NOS-dependent NO-formation is inhibited (e.g. after halogen exposure).

Furthermore, it is also clear that in mammals, nitrate is not an inert NO-oxidation product. Specifically, nitrate, which is formed endogenously and ingested in foods, is concentrated 10-20 fold (resulting in mM concentrations) in saliva²¹⁻²⁶. Here, nitrate is reduced to nitrite by oral bacteria expressing nitrate-reductases²⁷⁻³⁴. Produced nitrite is swallowed, where it can elicit NO-signaling in the acidic stomach, but also enters the plasma and provides substrate for NO-formation via nitrite-reduction in all tissues as outlined above^{24,35-37}. Thus, there exists a symbiotic relationship between oral microbes that use nitrate as a respiratory source, and the mammalian host, that is critical to NO-homeostasis.

C. Toxic Gas Exposure and Use of Nitrite

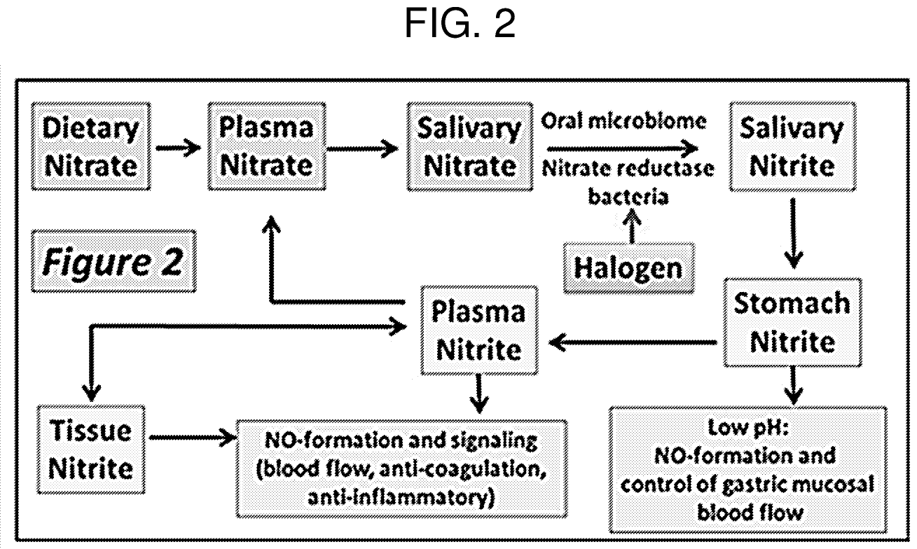

Cl₂, Br₂ and COCl₂ cause hemolysis in mice, rats, and guinea pigs^{5,7,38,39}. Moreover, transient drops in RBC count, likely due to hemolysis, can occur following human exposure to COCl₂⁴⁰. The present disclosure investigates the hypothesis that NR containing bacteria present on the posterior tongue mediate NO-bioavailability in vivo (FIG. 2). Data supporting this in humans includes the finding that dietary or therapeutic nitrate leads to temporal and sequential increase in plasma nitrate, salivary nitrate, salivary nitrite and plasma nitrite. In addition, nitrate administration stimulates NO-dependent signaling, as shown by lowering of blood pressure, improving blood flow, mitochondrial function and exercise capacity, inhibiting platelet aggregation, preventing end-organ inflammatory injury that temporally follow changes in salivary and plasma nitrite^{22,24,31,41-51}. Complementary studies in murine and rodent models show that nitrate ingestion also prevents inflammatory injury in all major tissues via nitrite and NO formation^{35,52-57}. Furthermore, the protective effects of dietary nitrate are prevented if oral bacteria are first depleted by chlorhexidine mouthwash compared to saline, and chlorhexidine mouthwash alone increases basal mean arterial pressure in humans^{41,58}. The corollary has also been shown, namely that feeding low-nitrate diets sensitizes mice to inflammatory injury⁵⁹.

The present disclosure further describes the use of nitrite as a treatment for inhalation chemical and smoke injuries. Low-dose nitrite therapy protects against injury caused by ischemia and inflammation (reviewed in^{18,60-64}) Underlying this therapeutic efficacy is nitrite-dependent repletion of NO-signaling^{62-63,65-75}. Developing a targeted efficacious therapy that is amenable to rapid administration in mass casualty scenarios is a focus of the CounterAct network. Importantly, nitrite can be stockpiled, is stable, is amenable to IM administration, and is an active ingredient in FDA-approved cyanide antidote kits. Nitrite has also undergone successful phase I/II studies for indications related to systemic/pulmonary hypertension and ischemia reperfusion injury^{46,76-80}.

### V. Embodiments

Embodiment 1. A method of treating a lung injury in a subject, wherein the lung injury is caused by inhalation of a chemical, inhalation of smoke, or SARS-CoV-2 infection, comprising administering to the subject a therapeutically effective amount of a nitrite salt, wherein the chemical is not chlorine, thereby treating the lung injury.

Embodiment 2. The method of embodiment 1, wherein the chemical comprises bromide, methyl bromide, mustard gas, nitrogen mustard, phosgene, phosgene oxime, diphosgene, phosphine, ammonia, bromine, methyl isocyanate, hydrogen chloride, osmium tetroxide, phosphorous, sulfuryl fluoride, lewisite, chloroacetophenone, chlorobenzylidenemalononitrile, chloropicrin, bromobenzylcyanide, dibenzoxazepine, or a combination or two or more thereof.

Embodiment 3. The method of embodiment 1 or embodiment 2, wherein the chemical comprises bromine or phosgene gas.

Embodiment 4. The method of embodiment 1, wherein the smoke is from a fire, exhaust fumes or an explosion.

Embodiment 5. The method of embodiment 1, wherein the subject has pneumonia resulting from SARS-CoV-2 infection.

Embodiment 6. The method of any one of embodiments 1-5, wherein the nitrite salt comprises sodium nitrite, potassium nitrite or arginine nitrite.

Embodiment 7. The method of any one of embodiments 1-6, wherein the therapeutically effective amount of nitrite salt is about 5 mg to about 600 mg.

Embodiment 8. The method of any one of embodiments 1-7, wherein the nitrite salt is administered as a formulation comprising the nitrite salt and an anti-caking agent.

Embodiment 9. The method of embodiment 8, wherein the concentration of the anti-caking agent in the formulation is at least 20 parts per million.

Embodiment 10. The method of embodiment 8 or embodiment 9, wherein the anti-caking agent comprises sodium bicarbonate.

Embodiment 11. The method of any one of embodiments 1-10, wherein the nitrite salt is administered by an intramuscular, intravenous, subcutaneous, oral or inhalation route.

Embodiment 12. The method of any one of embodiments 1-11, further comprising selecting a subject with a lung injury prior to administering the nitrite salt.

Embodiment 13. A formulation comprising a nitrite salt and an anti-caking agent, wherein the anti-caking agent is present at a concentration of at least 20 parts per million.

Embodiment 14. The formulation of embodiment 13, wherein the nitrite salt is sodium nitrite, potassium nitrite or arginine nitrite.

Embodiment 15. The formulation of embodiment 13 or embodiment 14, wherein the anti-caking agent comprises sodium bicarbonate.

Embodiment 16. A method of treating a disease or condition in a subject, comprising administering to the subject a therapeutically effective amount of the formulation of any one of embodiments 13-15, wherein the disease or condition is selected from a lung injury, pulmonary hypertension, heart failure, cardiogenic shock, hypertension, respiratory failure, a metabolic syndrome, diabetes, a lipid disorder, an endocrine disorder, a gastroenterological disorder, hypoperfusion, inflammation, cystic fibrosis and aging.

Embodiment 17. The method of embodiment 16, wherein the lung injury is caused by inhalation of a chemical, inhalation of smoke, or SARS-CoV-2 infection.

Embodiment 18. The method of embodiment 16, wherein the disease or condition comprises pulmonary hypertension and heart failure with a preserved ejection fraction (HFpEF).

Embodiment 19. The method of embodiment 16, wherein the heart failure comprises right-sided heart failure, left-sided heart failure or congestive heart failure.

Embodiment 20. The method of embodiment 16, wherein the hypertension comprises chronic hypertension, acute hypertension, urgency hypertension, emergency hypertension, prehypertension or a combination thereof.

Embodiment 21. The method of embodiment 16, wherein the respiratory failure comprises acute respiratory distress syndrome, respiratory failure from trauma or mechanical injury, respiratory failure due to pneumonia, respiratory failure resulting from an infectious disease, respiratory failure resulting from pulmonary edema, respiratory failure resulting from pulmonary embolism, respiratory failure due to infant respiratory distress syndrome, respiratory failure due to interstitial lung disease, or respiratory failure resulting from an autoimmune disease.

Embodiment 22. The method of embodiment 16, wherein the lipid disorder comprises hypercholesterolemia, hypertriglyceridemia, or both.

Embodiment 23. The method of any one of embodiments 16-22, further comprising selecting a subject with a lung injury, pulmonary hypertension, heart failure, cardiogenic shock, hypertension, respiratory failure, a metabolic syndrome, diabetes, a lipid disorder, an endocrine disorder, a gastroenterological disorder, hypoperfusion, inflammation, cystic fibrosis or aging prior to administering the formulation.

Embodiment 24. A method of enhancing cardiovascular performance in a subject, comprising administering to the subject a therapeutically effective amount of the formulation of any one of embodiments 13-15.

Embodiment 25. A method of providing a nutritional supplement to a subject, comprising administering to the subject the formulation of any one of embodiments 13-15.

The following examples are provided to illustrate certain particular features and/or embodiments. These examples should not be construed to limit the disclosure to the particular features or embodiments described.

### EXAMPLES

### Example 1: Effects of Toxic Gas Exposure on Oral Nitrate Reductase Activity and Microbiome

This example describes studies to evaluate the use of nitrite for treating chemical lung injury.

### Methods

Cl₂/Br₂/CoCl₂ Exposure: All exposures (whole body) were performed as previously described (Honavar et al., Am J Physiol Lung Cell Mol Physiol 307: L888-894, 2014). After exposure, mice were housed singly to limit microbiome impacting variables.

Nitrite and nitrite measurement: Nitrite and nitrate were measured by HPLC based Griess reaction (Eicom) as described^{6,12}. For plasma and saliva, nitrite and nitrate were extracted by methanol as described¹⁰³.

Bacterial colony growth: were assessed as described^{4,29}. Swab samples were serially diluted in tryptic soy broth, plated on agar plates and incubated for 12-24 h at 37° C. Defined colonies were counted by microscopy. Also, samples were diluted into nitrate medium (BD Gifco) to selectively assess denitrifying bacterial growth.

Sequencing: DNA was extracted from bacterial culture with DNA Isolation Kit (Zymo Research, cat. no. D6010). Amplicon library from individual samples was prepared by PCR using unique barcoded primers to amplify the hypervariable region V4 of the 16S rRNA. The PCR products from individual samples were run into and excised from agarose gel followed by purification using a commercial gel extraction kit. Purified PCR products were sequenced using the NextGen sequencing Illumina MiSeq platform. The microbiome analysis package, “Quantitative Insights Into Microbial Ecology”, was used for microbiome analysis as described¹⁰⁴.

### Measuring Oral NR Activity

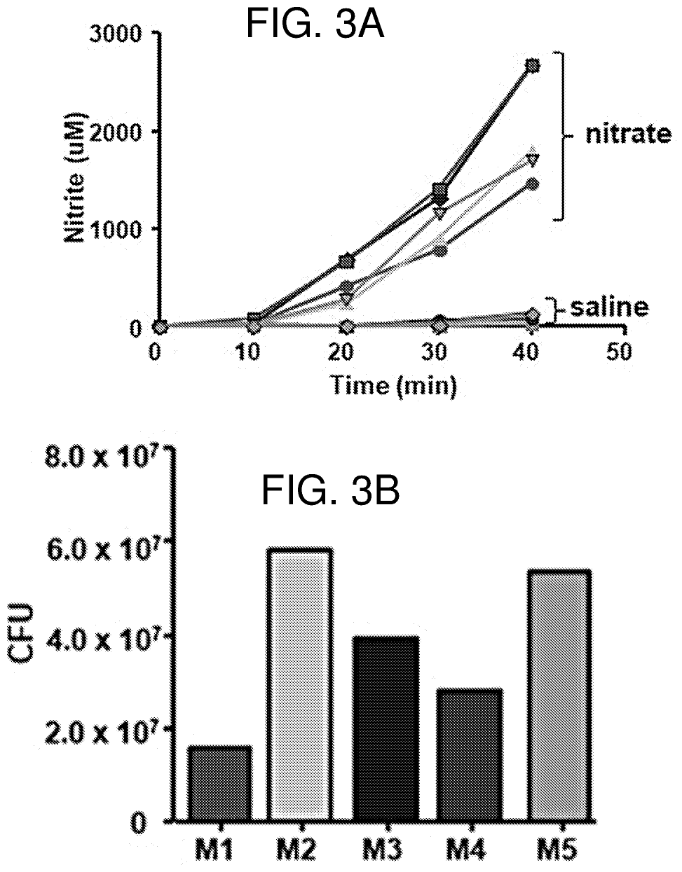

Nitrate-dependent modulation of NO-bioavailability, via the pathway described in FIG. 2, has been demonstrated in humans, rats and mice. This pathway mediates nitrate dependent stimulation of angiogenesis, prevention of pulmonary hypertension and inflammatory tissue injury, and its disruption increases basal blood pressure and platelet aggregation, underscoring the importance of this mechanism in regulating NO-bioavailability.^{35,52-57} This study tested the hypothesis that inhibition of NR activity by Cl₂, Br₂ or COCl₂ causes a loss of systemic NO-bioavailability. Few studies¹⁰⁰ have directly measured NR activity ex-vivo and none with mice. To enable direct measurement of NR activity, a protocol to assess NR activity from human and mouse samples was developed as described in Fanucchi et al. (Am J Respir Cell Mol Biol 46:599-606, 2012). Data with C57bl/6 adult male and female mice are shown in FIG. 3. Swabs of the distal tongue were cultured for 18 hours to increase bacterial numbers and NR activity was assessed by adding nitrate (or saline control) and measuring nitrite production (FIG. 3A). In parallel, bacteria were counted (FIG. 3B). FIG. 3C plots NR activity per CFU.

### Halogens Inhibit Oral NR Activity

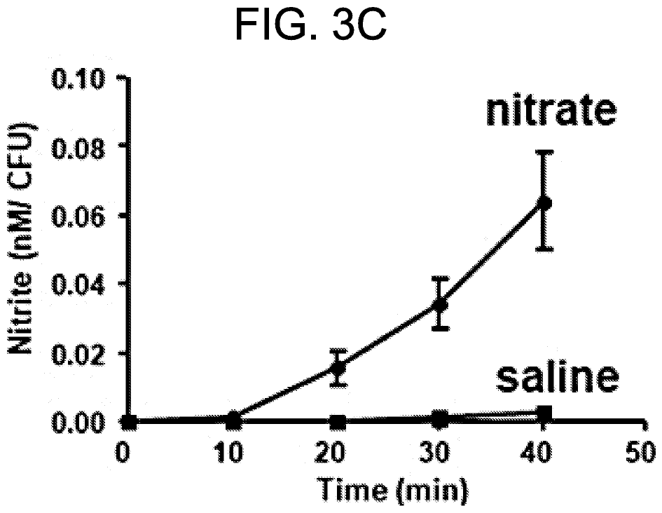

To test whether halogen gas exposure inhibits the enterosalivary nitrate-nitrite-NO pathway, mice were exposed to air or Br₂ gas (400 ppm, 30 minutes) and then brought back to room air. FIG. 4A shows that 24 hours after exposure, NR activity normalized per bacterial number was significantly inhibited compared to air exposed mice; however, bacterial number was not different (FIG. 4B). This result indicates that halogen gas exposure either selectively inhibits NR activity and/or alters the oral microbial composition favoring bacteria that do not express NR activity. FIG. 4C shows a similar inhibition of NR activity in a rat model of Br₂ exposure. Furthermore, FIG. 4D shows that Cl₂ gas also inhibited NR activity in rats, with inhibition persisting out to at least 48 hours post exposure. To control for possible selection artefacts associated with bacterial culturing, for FIGS. 4C-4D, NR activity was measured on whole tongues immediately after collection and without any post-sampling culturing.

### Oral Nitrate Reductase Activity Ex Vivo

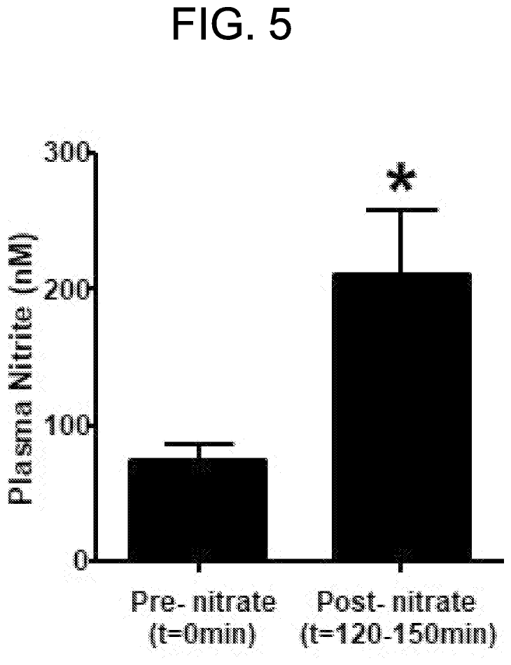

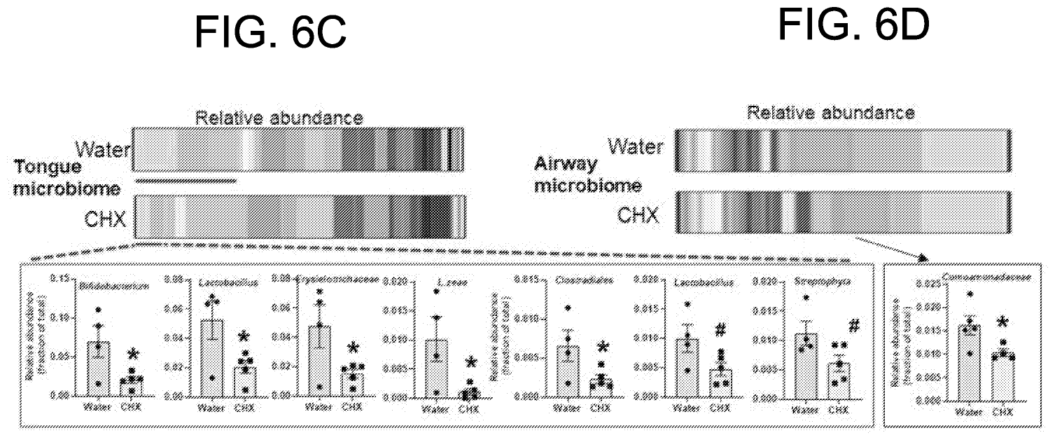

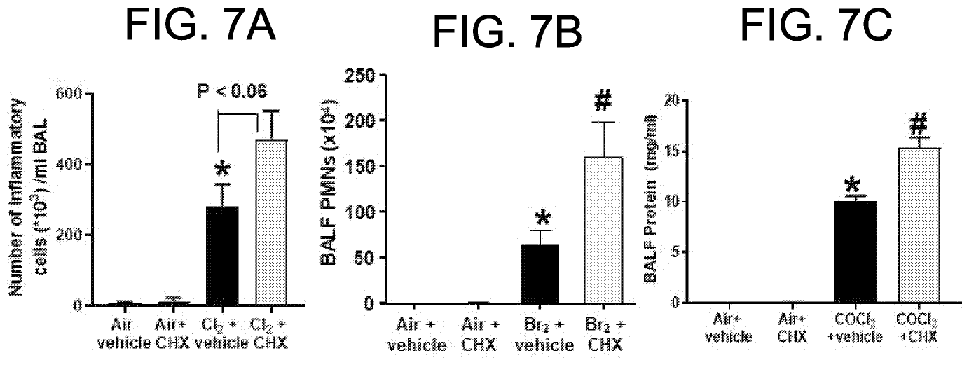

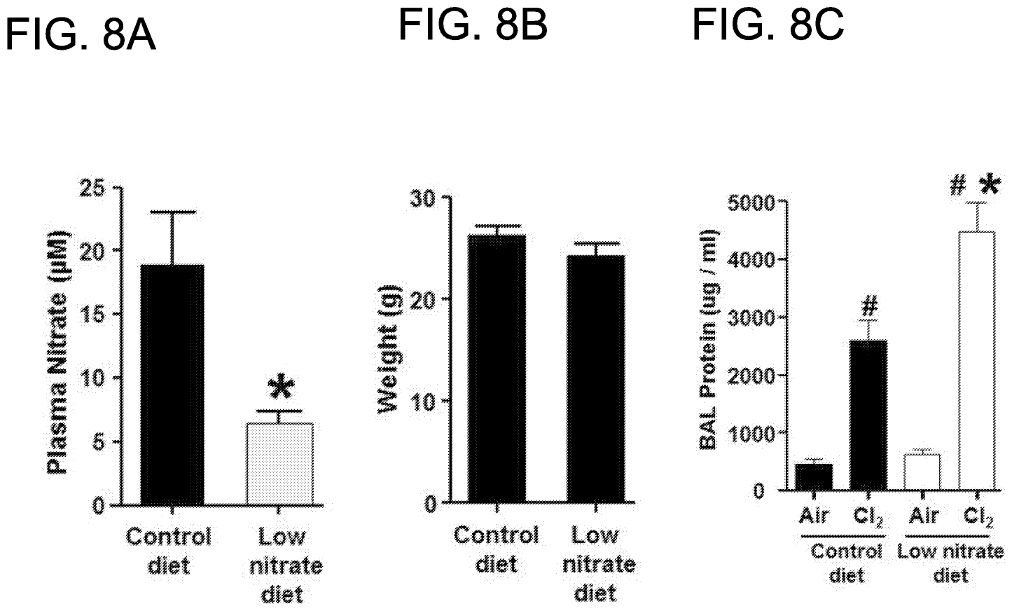

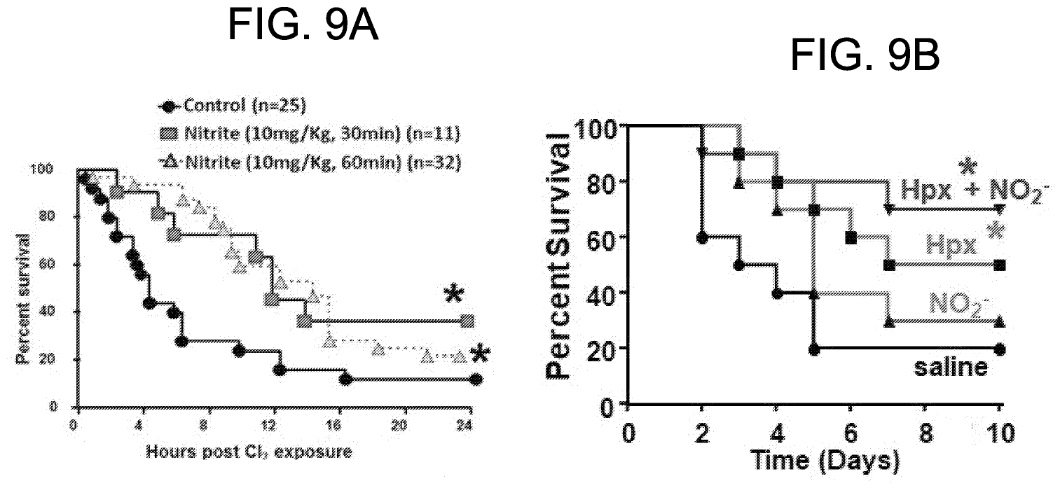

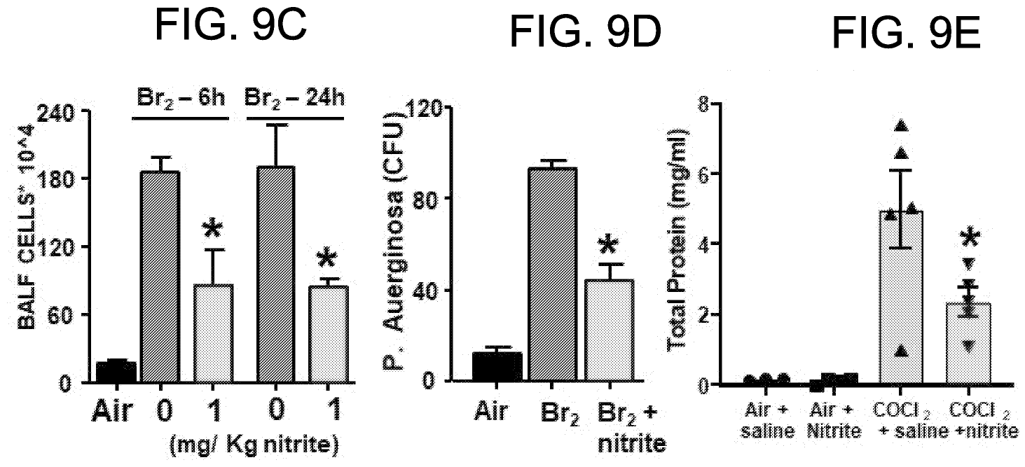

The following study is carried out to determine if Cl₂, Br₂ or COCl₂ gas inhibit oral nitrate reductase activity ex vivo. Eight-week old male and female C57Bl/6 mice are exposed to room air, C12 (400 ppm for 30 minutes or 600 ppm for 30-45 minutes), Br₂ (400 or 600 ppm, 30-45 minutes) or COCl₂ (10 ppm or 20 ppm, 10 minutes), and then brought back to room air. Exposure conditions are based on prior publications¹⁴⁻⁷ and FIG. 9 below, showing that these conditions result in hemolysis and post-exposure lung injury at lower doses, and ˜50% mortality over 24 hours at the higher doses. The ability to expose mice to COCl₂ is also demonstrated in FIG. 7 and FIG. 9. All exposures are performed between 7:30-9:30 am to minimize differences due to circadian rhythm. After exposure, mice are brought back to room air and housed individually in cages, under identical conditions, to minimize variables that could affect microbiome diversity. At 6 hours, 24 hours, 48 hours, 72 hours, 4 days and 7 days post-exposure, dorsal mouse tongues are swabbed, cultured for 18 hours, and NR activity and bacterial number are assessed. If activity does not recover by 7 days, additional times over the following 2 weeks are tested. Non-invasive sampling allows for longitudinal (paired) assessment of changes in the oral microbiome. However, this also requires culturing of tongue swabs for 18 hours, which may introduce a selection bias. To control for this, conditions where maximal inhibition is observed are selected, and whole tongues are excised past the buccal pad and NR activity measured immediately at collection. This protocol provides sufficient material to measure NR activity without the need to culture swabs. Comparing data from swab-culture with whole tongues also tests if Cl₂ effects are due to inhibition of bacterial proliferation versus during exposure cytotoxicity. It is expected that toxic gas exposure will inhibit tongue NR activity.

### Oral NR Activity In Vivo

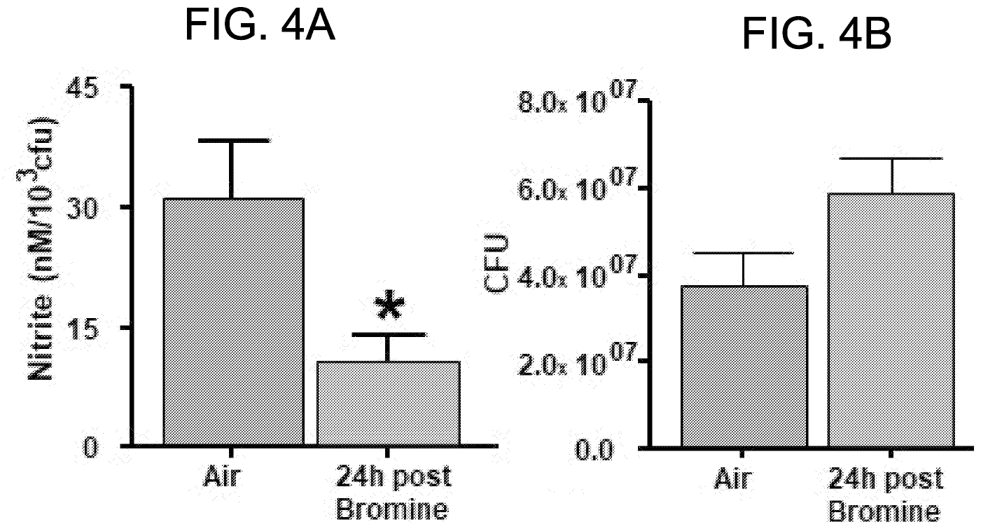

The following study is carried out to determine if Cl₂, Br₂ or COCl₂ inhibits oral NR activity in vivo. To assess whether inhibition of oral NR activity results in a loss of NO-bioavailability, the pathway outlined in FIG. 2 is interrogated. Conditions in which each gas maximally inhibits NR activity (expected to be 24-48 hours) are used. To assess NR activity, blood is sampled 2 hours pre, 2 hours post (by tail vein, 100 μl) and 4 hours post (sacrifice) exogenous saline or nitrate (100 mg/Kg, one-time IM injection in 50 μl volume) administration. Plasma nitrite, nitrate and cGMP, a marker of NO-dependent sGC activation, are measured. IM injection of nitrate increases circulating nitrate <5 minutes, and nitrate-dependent changes in nitrite and NO-signaling occurs within 4 hours of administration, with peak changes around 2-3 hours^{46,47}. FIG. 5 demonstrates the functionality of oral NR reductase activity in air-breathing mice; plasma nitrite levels increase 2.5 hours after nitrate addition. It is expected that compared to saline, nitrate will temporally increase plasma nitrite and cGMP, with these changes being blunted in mice exposed to toxic gases. Oral bacteria are required for nitrate reduction^{23,24,29-31,41,43,44,46} in vivo, therefore this assay reports on acute effects of exogenously added nitrate and reflects activity of oral nitrate-reducing bacteria. This study design will avoid confounding aspects of basal differences in plasma nitrite and nitrate that may exist due to Cl₂, Br₂ or COCl₂ exposure per se.

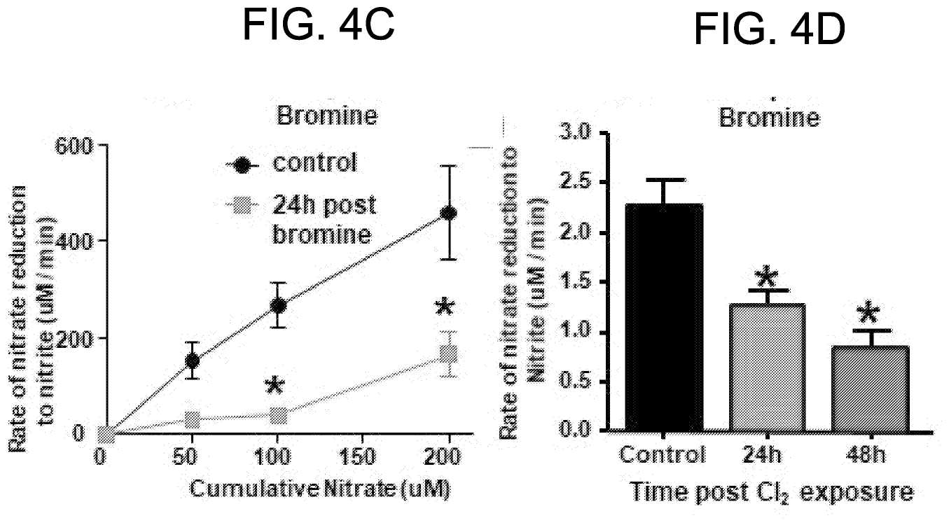

To further isolate the enterosalivary system, the study includes a group in which mice are treated with L-NAME to inhibit all nitric oxide synthases 1 hour prior to testing of nitrate-dependent effects. In addition, and to complement these studies, effects of Cl₂, Br₂ or COCl₂ exposure on lung and aortic NOS activity are determined using enzyme activity assays, functional assays (aortic ring dilation) and expression (western blotting), as described previously^{6,12} Finally, a positive control group is included to ensure loss of oral NR activity. Specifically, the posterior tongues of air exposed mice are treated with water or chlorhexidine as described in Example 2 below (see FIG. 6 for data supporting this model). It is expected that toxic gas exposure will inhibit nitrate-dependent increases in plasma nitrite in vivo.

### Mechanism for Inhibiting Oral NR Activity

The following studies are carried out to determine how Cl₂, Br₂ or COCl₂ inhibits oral NR activity. These studies will determine whether decreased NR activity post Cl₂, Br₂ or COCl₂ exposure is due to lower bacterial number, and/or due to inhibition of NR activity without bacterial killing (i.e. lower specific activity). The studies will also profile the oral nitrate-reducing microbiota and assess if this is reprogramed by toxic gas exposure.

Mice are exposed to air or Cl₂, Br₂ or COCl₂ using the dose that each resulted in maximal inhibition of NR activity. Mice are brought back to air and tongues are collected at various times post-exposure (6 hours, 24 hours, 48 hours, 72 hours, 4 days, 7 days) to assess acute and chronic changes in microbiome diversity. A pre-collection (1 day prior to Cl₂ exposure) time point is also included. Tongues are scraped to collect resident bacteria. Since qPCR measures both live and dead cell derived DNA, bacterial number is determined using colony growth assays. NR activity is also determined and normalized to bacterial number to assess specific activity.

To profile the oral nitrate-reducing microbiota, total DNA is isolated, followed by PCR amplification of the V4 region of the rRNA gene and NextGen sequencing of the PCR product to assess microbial diversity and taxonomic identification. The goal is to determine how toxic gas exposure changes the oral microbial community and evaluate whether it is reprogrammed with specific attention to communities where oral nitrate-reducing activity resides. To this end, the distal dorsal surface of the tongue where NR bacteria reside are sampled (see Fanucchi et al., Am J Respir Cell Mol Biol 46:599-606, 2012). To compare, the proximal (front) of the tongue is also sampled to determine whether the effects of each gas on nitrate-reducing bacteria is dependent on lingual location.

Additionally, little is known on how oral microbial diversity is affected by diet, housing and other factors including mice being coprophagic. Therefore, mice are housed one per cage after air or toxic gas exposure to limit possible variability associated with these factors. Finally, samples of lung tissue, upper GI and lower GI tracts, and fecal matter are samples and microbiome sequencing is performed to compare with the composition of oral microbes. This will determine whether toxic gas exposure effects on the microbiome are limited to the oral cavity. Data shown in FIG. 6C demonstrate feasibility of the proposed studies.

Finally, due to differences between mouse and human microbiomes, and to assess whether the findings are translatable to humans, swabs from the back of the tongue are collected from ten male and ten female healthy age-matched human volunteers, cultured as described in methods below for 18 hours and then exposed in vitro to air, Cl₂, Br₂ (each at 0, 200, 400, 600 ppm) for 0-30 minutes, or COCl₂ (10 or 20 ppm, 0-15 minutes). At various times post-exposure, bacterial number, diversity and NR activity are determined.

### Example 2: Effects of Depleting the Oral Microbiome and Dietary Nitrate on Toxic Gas-Dependent Injury

The data described in this example demonstrate that factors negatively affecting the enterosalivary NR system, namely low nitrate in the diet and/or lower levels of nitrate-reducing bacteria, pre-dispose mice to the injurious effects of hemolysis resulting in greater lung injury and toxicity after exposure to Cl₂, Br₂ or COCl₂.

First, the effects of selective depletion of the oral microbiome on Cl₂, Br₂ or COCl₂ toxicity was tested. For these studies, a method was developed for inhibiting NR activity in the oral cavity. C57Bl/6 mice were briefly anesthetized using ketamine/xylazine and a small volume (10 μl) of chlorhexidine (0.2%) or water (control) applied topically to the back of the tongue twice per day for 7 days. This protocol was optimized to ensure only the posterior tongue was exposed to chlorhexidine, and showed that compared to vehicle control, NR activity was inhibited by ˜70% (FIG. 6A) and plasma nitrite decreased by 50% (FIG. 6B), consistent with an inhibition of the enterosalivary NR system. Bacterial abundance and diversity were measured by 16S sequencing on posterior tongue swabs lung (BALF) sampling. For these analyses, the 25 most abundant bacteria, in control mice, in either tongue or lung compartments were determined and the effects of chlorhexidine on these were assessed. FIGS. 6C-6D show relative bacterial abundance in water (vehicle) and chlorhexidine treated mice, and plots the bacteria that were significantly different between chlorhexidine and control mice. For the sake of clarity, bacteria with non-significant changes were not listed. On the tongue, chlorhexidine decreased abundance of 7 bacteria (5 at p<0.05 and 2 at p<0.07 level) by >50%; no bacteria increased in abundance in this compartment. Of these seven, four are known NR containing suggesting loss of these may mediate lower oral NR activity (FIGS. 6A-6B). FIG. 6D plots changes in the lung microbiome. Only one bacterial species was altered, decreasing by a more modest ˜30%.

In the next series of experiments, after chlorhexidine treatment, mice were exposed to Cl₂, Br₂ or COCl₂ and acute lung injury (ALI) was assessed. FIGS. 7A-7C show that chlorhexidine alone had no effect on lung injury. However, chlorhexidine significantly increased the severity of Br₂ and COCl₂by 1.5-2.5 fold, with close to significant effects observed with Cl₂ (p=0.06).

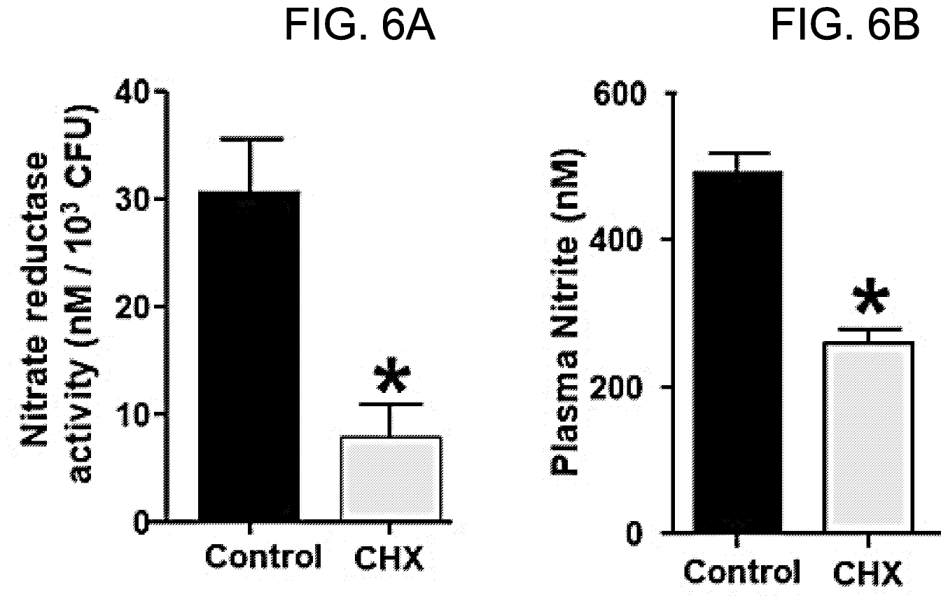

Next, mice were placed on control diet (that contains nitrite+nitrate), an isocaloric low nitrate diet (50% lower nitrite+nitrate content); low nitrate diets were formulated to ensure similar nutritional content between groups as described⁵⁹. FIG. 8A shows low nitrate diet feeding for 2 weeks decreased plasma nitrite levels consistent with a deficit in the enterosalivary nitrate-reduction pathway. No differences in weight (FIG. 8B), food or water consumption between groups was observed. FIG. 8C shows that mice fed low nitrate diet had higher levels of protein in their BAL 24 hours post C12 exposure. Collectively, the data shown in FIGS. 7-8 indicate that oral microbes modulate halogen and COCl₂-stimulated ALI and individuals with low dietary nitrate or low oral NR activities are more vulnerable to toxicity.

Determine if the Oral Microbiome Limits Cl₂, Br₂ or COCl₂ Gas Toxicity

Chlorhexidine (10 μl, 0.2% in water) or vehicle control is applied topically on the posterior tongues of C57Bl/6 male and female mice 2x per day, for 7 days, to inhibit oral NR activity. Effectiveness of chlorhexidine treatment is assessed by measuring oral NR activity, microbial viability and diversity, and plasma nitrite and nitrate. Then mice are exposed to air, Cl₂ (400 ppm, 30 minutes) Br₂ (400 ppm, 30 minutes) or COCl₂ (10 ppm, 10 minutes) and the following variables are measured at 6 hours, 24 hours, and 48 hours post-exposure (i) ALI, (ii) airway reactivity to methacholine, (iii) eNOS-dependent dilation in aortas (which is inhibited in Cl₂ exposed mice¹²), and (iv) susceptibility to P. Aeruginosa infection. Separately, mice are exposed to C12 (600 ppm, 45 minutes), Br₂ (600 ppm, 45 minutes), or COCl₂ (20 ppm, 10 minutes) and 24 hour survival is determined. Previous studies with COCl₂ have established ˜50% mortality for these gases using these exposure conditions^{1,4-7}. It is expected that mice in which their oral NR activity has been depleted will display a greater severity of post-toxic gas exposure toxicities.

Determine if Low NOx Diet Predisposes to Greater Cl₂, Br₂ or COCl₂ Induced Toxicity

Eight week old C57bl/6 male and female mice are fed regular chow, an isocaloric low nitrate diet, or an isocaloric low nitrate diet with nitrate supplemented drinking water, for 14 days prior to exposure to Cl₂, Br₂ or COCl₂ using conditions that will allow testing of acute lung injury and 24 hour survival end points, as described above. Nitrate in the drinking water is titrated to restore circulating levels observed in the normal chow group. Consumption of food, water, and animal weight, oral NR activity and plasma nitrate and nitrite levels are measured every 3 days. It is expected that low NOx diet groups will show greater lung and systemic vascular injuries compared to normal diet, and that nitrate in the drinking water will prevent this. It is further expected that these results will be highly significant due to the relatively low nitrate, and hence lower plasma nitrite levels in individuals consuming typical western and nutrient poor diets.

Determine the Effects of Low NOx Diet and Oral-Microbial Disruption on Cl₂, Br₂ or COCl₂ Induced Toxicity

The studies above test whether selective depletion of oral cavity dorsal lingual bacteria, or nitrate from the diet exacerbate toxic gas injury. In this study, it will be determined if the combination of these interventions leads to further increases in injury. C57Bl/6 male and female mice are placed on normal chow, low nitrate diet or low nitrate diet+nitrate in the drinking water for 2 weeks. Over the last 7 days, mice are further split into groups to receive vehicle or oral chlorhexidine and then Cl₂, Br₂, COCl₂ lung injury (using one end-point showing greatest sensitivity) and mortality determined. It is expected that injury will be most severe in combined low NOx and chlorhexidine groups, and that protective effects of nitrate in the drinking water will be lost in mice treated with chlorhexidine due to an inability to reduce nitrate to nitrite.

Determine the Mechanism(s) by which Inhibition of the Enterosalivary Nitrate-Reduction System Increases Cl₂, Br₂, COCl₂ Toxicity

NO is an antioxidant, and an anti-inflammatory agent. It is hypothesized that exacerbated injury in mice with an inhibited enterosalivary NR system is due to a greater magnitude of damage along the pathway of Cl₂, Br₂, COCl₂ derived oxidized or halogenated lipids, hemolysis, and free hemoglobin/free heme-dependent oxidative and inflammatory injury to the lung. To test where along this pathway NO exerts protective actions, conditions will be used that are expected to show maximal exacerbation of injury caused by each gas and the following will be tested:

i) Measure Cl-lipids, Br-lipids or oxidized lipids in the lung and plasma immediately following, and 6 hours and 24 hours post-exposure.

ii) Measure plasma and BAL free hemoglobin, heme, non-transferrin bound iron (NTBI), hemopoexin and haptoglobin. A spectral deconvolution assay is used for Hb and heme¹⁰⁷. In addition, it is tested whether RBC fragility to osmotic and mechanical stress is enhanced in mice with inhibited NR activities; prior studies have shown NO improves RBC deformability¹⁰⁸.

iii) Measure indices of oxidative and inflammatory injury including F₂-isoprostanes, ferrylHb and protein oxidation; these parameters report on hemolysis-derived products and lipid peroxidation and are measured using protocols described in published studies^{109,110}. Heme is also a TLR4 ligand and downstream inflammatory pathways are inhibited by NO⁹⁹. To test this, NF-κB signaling and candidate target genes including ICAM-1 and VCAM1 are tested by Western blotting. It is hypothesized that heme-dependent TLR4 activation will be greater in low nitrate diet and chlorhexidine treated mice. Data suggest heme-mediated cellular injury via inducing mitochondrial DNA damage. This is assessed by measuring circulating mitochondrial DNA fragments.

iv) Test whether low nitrate diet and chlorhexidine treatment sensitizes mice to exogenous hemoglobin and heme-dependent ALI, and increased P. Aeruginosa infection independent of toxic gas exposure. Specifically, oxyhemoglobin (0-50 μM), methemoglobin (0-50 μM) or heme (0-50 μM) are administered intratracheally as previously described” and ALI measured (see below) at 6 hours, and compared to vehicle controls. End points related to oxidative and inflammatory stress are also measured.

### Methods

Acute lung injury (ALI) was assessed by measuring: (i) BAL changes in protein, inflammatory cells and cytokines^{112,113}. Mice were euthanized with IP ketamine/xylazine (100 and 10 mg/kg body weight) and a 3 mm endotracheal cannula was inserted in their tracheas. Lungs were lavaged with 2 ml of 0.9% NaCl three times. Recovered lavage fluid was centrifuged immediately at 300 g, 10 minutes to pellet cells. (ii) Wet:dry weight ratios: bloodless lung wet/dry weight ratios were determined as previously described¹¹³. (iii) Arterial blood gases, oxygen saturation and pH were measured as previously described. (iv) Airway/alveolar morphological evaluation of injury to the alveolar and mid-upper airways was made as recently described¹¹². Specific measurements included assessment of apoptosis and necrosis, and pathology scoring, all performed in a blinded manner

Airway reactivity and chronic lung injury was measured under basal and methacholine challenge by Flexivent¹¹².

Systemic endothelial function was assessed using ex vivo vasodilation studies and eNOS expression/activity measurements in isolated aorta as described^{12,66}. Each experiment determined vessel contractility to increasing doses of phenylephrine followed by assessment of eNOS-dependent vasodilation using acetylcholine, and then endothelium independent vasodilation determined using Mahma nonoate.

Cl-/Br-lipids and oxidized lipids were measured as previously described^{3,105,114}. Sample processing protocols that avoid artefactual oxidation were used including using metal chelators, BHT and limiting light exposure.

Hemolysis-derived mediators: Free hemoglobin, heme, non-transferrin bound iron were measured according to standard procedures. Haptoglobin and hemopexin were measured by ELISA as described^{96,107,115}.

### Example 3: Effects of Post-Exposure IM (Intramuscular) Nitrite Alone or in Combination with Hemopexin (a Heme-Scavenger) on Cl₂, Br₂ and COCl₂ Toxicity

IM injection of nitrite, post-Cl₂ gas exposure, decreases ALI and reactive airways in mice and rats¹⁻³. Nitrite is chemically stable, can be stockpiled, and is amenable to administration in mass-casualty scenarios. Since hemolysis is a common injury causing pathway for Cl₂, Br₂ and COCl₂, it was hypothesized that nitrite may be protective against Br₂ and COCl₂ as well. The studies in this example test whether a single IM injection of nitrite can afford protection against Br₂ and COCl₂ toxicity, and whether the efficacy of nitrite-cytoprotection is improved by combining with other therapeutics, specifically hemopexin, that also protect against hemolysis-dependent injury.

FIG. 9A shows that IM nitrite given to mice post-Cl₂ gas exposure improves acute survival, with the greatest survival benefit observed up to 18 hours post-exposure. It is proposed that this property of nitrite is useful for increasing the time window to allow transport of exposed individuals to primary care settings and administration of secondary more targeted therapeutics such as hemopexin. Rationale for testing Br₂ is provided by FIG. 9B, which shows that a single TM injection of nitrite 30 minutes post-Br₂ exposure decreases inflammatory cell accumulation in the BAL at 6 hours and 24 hours. Furthermore, nitrite decreases susceptibility to P. aeruginosa infection (FIG. 9C), similar to that observed with hemopexin. Failure to kill pathogens is heme-dependent, and suggests that nitrite either prevents heme formation and/or heme-dependent toxicity. This also raises the question of whether a combination of nitrite (an anti-inflammatory and anti-oxidant) and hemopexin (which scavenges heme) afford additive or synergistic protection. FIG. 9D presents data (n=10) showing that the combination of nitrite and hemopexin improves survival post Br₂ more effectively than nitrite or hemopexin alone. Hemopexin alone improved survival (p=0.08) and nitrite improved survival early (<3d) post-exposure (p=0.08). These data indicate that together, nitrite and hemopexin improve either countermeasures' therapeutic profile by allowing the use of lower concentrations, and/or resulting in greater and longer-lasting protection. Furthermore, data in FIG. 9E shows that IM nitrite, administered 30 minutes post-exposure, limits lung injury when administered post COCl₂ exposure.

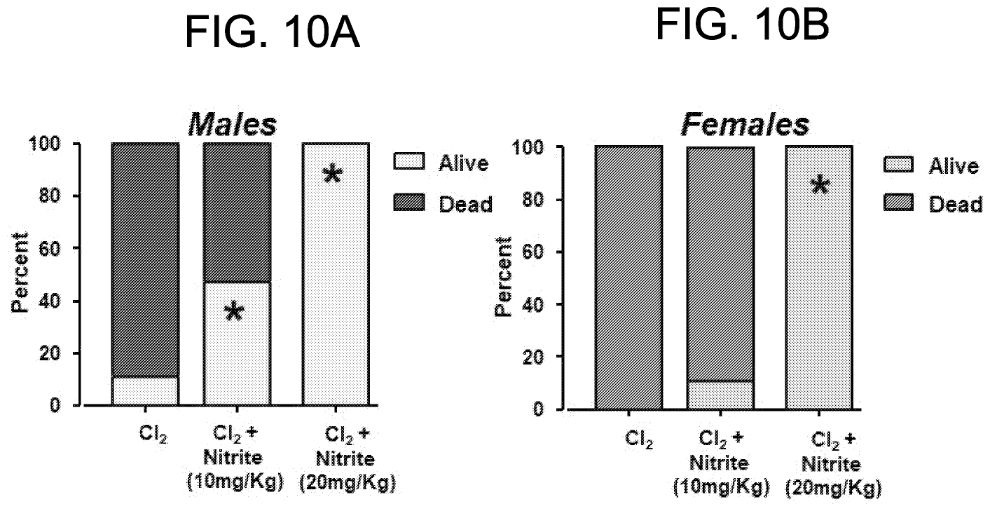

Additional studies in this example will determine if nitrite-therapy is effective in susceptible populations (e.g., mice with decreased NO bioavailability), and test whether efficacy is similar in males vs. females. The latter is an important consideration as studies show nitrite is a more potent anti-platelet effector in male subjects⁴⁴. Consistent with this concept, it was observed that higher doses of nitrite are required to improve post-Cl₂ gas survival in female mice compared to male mice. FIGS. 10A-10B show that both 10 mg/Kg and 20 mg/Kg nitrite improved survival of male mice but only the highest dose was protective in females.

Determine if Post-Exposure IM Injection of Nitrite Protects Against Cl₂, Br₂ or COCl₂ Induced Toxicity

This study focuses on two areas: (i) Optimizing nitrite dosing and timing of nitrite administration post-toxic gas exposure, and (ii) optimizing these parameters in males vs. females exposed to Br₂ or COCl₂. Testing of nitrite against Cl₂ has been completed (see¹⁻³ and FIGS. 8-10), thus the present study focuses on Br₂ or COCl₂. The experimental approach exposes male and female mice to Br₂ or COCl₂ gas using two exposure regimens; a sublethal exposure that allows measurement of lung injury (including ALI P. aeruginosa infection), and a lethal exposure protocol that results in 50-75% mortality over 24 hours. Sublethal and lethal exposure protocols outlined Examples 1 and 2 are used. A single IM injection of nitrite at 0, 0.1, 1, 10, 20 mg/Kg is administered at 0.5, 1 or 2 hours after exposure to determine the threshold time, post-exposure, after which nitrite no longer affords protection. Sub-lethal experiments are conducted first to determine optimal time and nitrite dose and then this condition is tested using the lethal exposure protocol. Nitrite dosing strategies are based on data (FIG. 9) showing that 1 mg/Kg IM decreased BAL neutrophil by 50%; therefore doses above and below this are used. This also allows for determination of upper doses of nitrite that can be used before potential toxicities are observed (previous studies demonstrate the U-shaped dependence of nitrite cytoprotection in ischemia-reperfusion models¹⁶). Moreover, this will allow for determination of whether optimal dose ranges differ between females and males. It is expected that to observe similar degrees of protection, higher nitrite doses will be required for females compared to males.

Finally, using conditions defined in Examples 1-2 that show the most severe ALI (e.g., in low NOx diet and chlorhexidine treated mice), nitrite therapy dosing studies are performed to assess whether this will be different in populations more susceptible to injury due to decreased basal NO-bioavailability.

Determine Mechanisms by which Nitrite Protects Against Br₂ or COCl₂ Toxicity

This study aims to define the mechanism by which nitrite therapy is protective. Understanding the mechanism(s) will aid development of nitrite-therapy and provide insights into why nitrite is more potent in males compared to females. In Example 1, it was hypothesized that loss of NO exacerbates inflammatory and oxidative stress. In this study, it is expected that nitrite-reduction to NO prevents this 16,17,19,23,116

Using the nitrite dose that conferred maximal protection against Br₂, and COCl₂, studies will assess (i) whether nitrite-mediates protection via anti-inflammatory mechanisms and measure expression of adhesion molecules (E-selectin, VCAM-1, ICAM-1 and P-selectin) and pro-inflammatory cytokines (IL1β, IL-6, IFN-γ, MCP-1 and TNFα) in the lungs by ELISA and/or Western blotting. Also, PMN depletion prevents protection mediated by nitrite towards Cl₂ gas toxicity¹. Thus, PMN is depleted using IP injection with 200 μg of anti Ly-6G (clone 1A8) (Bxcel: cat# BE0075-1) and compared to IgG2a Isotype control (Bxcel: cat# BE0089) 24 hr prior to Br₂ or COCl₂ gas exposure. (ii) Whether nitrite-mediated protection occurs via anti-oxidant effects: This is tested by measuring markers of reactive oxygen, nitrogen and bromine species in the lung and plasma including F₂-isoprostanes, protein carbonyl, 3-nitrotyrosine and bromotyrosine adducts¹¹⁴. (iii) If nitrite-mediated protection occurs via preventing cell death. Apoptosis is determined in the lung by TUNEL staining. Anti-apoptotic effect is verified by measuring caspase-3 activity in the lung sections by immunofluorescence using anti-active caspase-3 antibodies. (iv) Determine NO-metabolite profile and pharmacokinetics after nitrite administration. Lungs, aorta and blood are collected for assessment of NO-metabolites (nitrite, nitrate, S-nitrosothiols, C—N-nitroso and heme nitrosyl). the pattern of NO-metabolites provides insights into nitrite-reactivity associated with cytoprotection and therefore insights into mechanism. Tissue is collected and immediately processed in ‘stabilization solutions’, that are required to stabilize different NO-metabolites and prevent artefactual inter-conversion between them¹⁰³ (v) Test if the protective effects of nitrite are meditated by NO formation. Mice are treated with the NO-scavenger C-PTIO (1 mg/Kg) or saline vehicle immediately (1-2 minutes) before nitrite administration. Control studies include addition of C-PTIO alone to air, Br₂ or COCl₂ gas exposed mice. (vi) Determine effects of nitrite on halogenated lipids. It is possible that nitrite, via stimulation of blood flow and perfusion facilitates halogenated lipid clearance and thereby decreases the effects of these species as post-exposure injury mediators. Br- and oxidized lipids are measured by GC-MS. (vii) Determine effects of nitrite on heme and iron. Hemopexin protects against increased infection risk post Br₂ exposure, implicating a role for free heme. Free heme and non-transferrin bound iron levels, hemopexin and haptoglobin are measured to specifically assess effects of nitrite on hemolysis.

Assess the Therapeutic Efficacy of Nitrite in Combination with Hemopexin

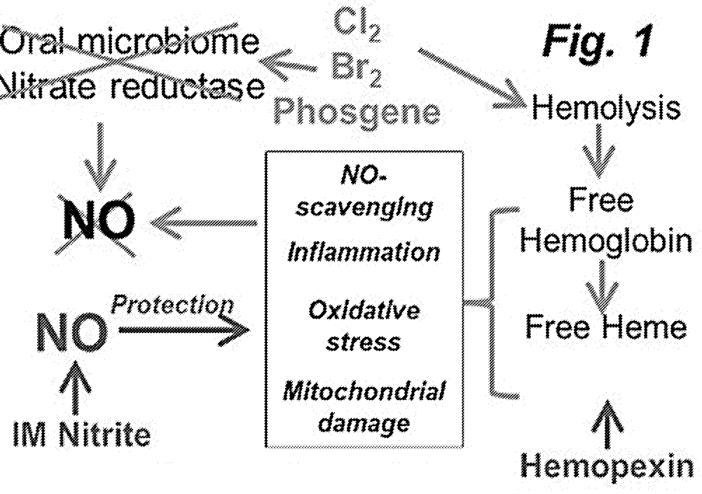

Data show that individually, nitrite or hemopexin administered post-Cl₂, Br₂, or COCl₂ exposure protects against ALI and P. aeruginosa infection (FIG. 9). These distinct countermeasures target different steps of the hemolysis-pathway (FIG. 1) and support the hypothesis that hemolysis is a common injury causing pathway for Cl₂, Br₂ and COCl₂ toxicity. This study will test two specific questions with the goal to optimize countermeasure efficacy. First, studies are performed to determine whether the combination of nitrite and hemopexin (which scavenges free heme) or nitrite afford additive or synergistic protection, and specifically whether the use of these together allow for using lower doses of each countermeasure to achieve similar protective effects. This is an important consideration to limit possible toxicities associated with high concentrations of each countermeasure alone. Data supporting these experiments is shown in FIG. 9D and in published reports demonstrating nitrite and hemopexin alone protect against ALI in a mouse model of trauma-hemorrhage⁹⁶. Rationale for testing nitrite is provided by published data showing that nitrite improves mitochondrial function in part by inhibiting mitochondrial derived reactive oxygen species^{119-122.} Second, studies are performed to determine if immediate IM nitrite therapy increases the operating window for delayed hemopexin or mitochondrial targeted therapy. To test the first question, male and female mice are exposed to Cl₂, Br₂ or COCl₂ gas (using sublethal or lethal exposure doses described above). Nitrite and hemopexin are then administered alone or together, 1, 3 or 6 hours after exposure. An additive effect leads to 50% protection, whereas synergistically leads to >50% protection. Next, starting at nitrite and hemopexin doses that alone show 50% protection, the dose of each compound is lowered by half and single and additive effects are tested. This tests whether lower doses of each countermeasure in combination provides protection equal to higher doses of each therapeutic when administered alone. To test the second question, male and female mice are exposed as described above and administered vehicle or nitrite at the lowest effective dose by a single IM injection at 30 minutes. Hemopexin is then administered at either 1 hour, 3 hours, or 6 hours post-exposure and ALI and susceptibility to infection is measured 24 hours after exposure, and survival over 10 days. Since nitrite and hemopexin target distinct steps of the hemolysis pathway (FIG. 1) it is expected that the combination of countermeasures will result in therapeutic synergy and allow delayed administration of hemopexin.

### REFERENCES

1. Kapil V, Weitzberg E, Lundberg J O, Ahluwalia A. Clinical evidence demonstrating the utility of inorganic nitrate in cardiovascular health. Nitric Oxide 2014; 38:45-57.
2. Kapil V, Haydar S M, Pearl V, Lundberg J O, Weitzberg E, Ahluwalia A. Physiological role for nitrate-reducing oral bacteria in blood pressure control. Free Radic Biol Med 2013; 55:93-100.
3. Larsen F J, Ekblom B, Sahlin K, Lundberg J O, Weitzberg E. Effects of dietary nitrate on blood pressure in healthy volunteers. N Engl J Med 2006; 355:2792-3.
4. Velmurugan S, Gan J M, Rathod K S, et al. Dietary nitrate improves vascular function in patients with hypercholesterolemia: a randomized, double-blind, placebo-controlled study. Am J Clin Nutr 2015.
5. Lara J, Ashor A W, Oggioni C, Ahluwalia A, Mathers J C, Siervo M. Effects of inorganic nitrate and beetroot supplementation on endothelial function: a systematic review and meta-analysis. Eur J Nutr 2015.
6. Kapil V, Khambata R S, Robertson A, Caulfield M J, Ahluwalia A. Dietary nitrate provides sustained blood pressure lowering in hypertensive patients: a randomized, phase 2, double-blind, placebo-controlled study. Hypertension 2015; 65:320-7.
7. Velmurugan S, Kapil V, Ghosh S M, et al. Antiplatelet effects of dietary nitrate in healthy volunteers: involvement of cGMP and influence of sex. Free Radic Biol Med 2013; 65:1521-32.
8. Bahra M, Kapil V, Pearl V, Ghosh S, Ahluwalia A. Inorganic nitrate ingestion improves vascular compliance but does not alter flow-mediated dilatation in healthy volunteers. Nitric Oxide 2012; 26:197-202.
9. Kapil V, Milsom A B, Okorie M, et al. Inorganic nitrate supplementation lowers blood pressure in humans role for nitrite-derived NO. Hypertension 2010; 56:274-81.
10. Webb A J, Patel N, Loukogeorgakis S, et al. Acute blood pressure lowering, vasoprotective, and antiplatelet properties of dietary nitrate via bioconversion to nitrite. Hypertension 2008; 51:784-90.
11. Presley T D, Morgan A R, Bechtold E, et al. Acute effect of a high nitrate diet on brain perfusion in older adults. Nitric Oxide 2011; 24:34-42.
12. Jones A M. Influence of dietary nitrate on the physiological determinants of exercise performance a critical review. Appl Physiol Nutr Metab 2014; 39:1019-28.
13. Wylie U, Bailey S J, Kelly J, Blackwell J R, Vanhatalo A, Jones A M. Influence of beetroot juice supplementation on intermittent exercise performance. Eur J Appl Physiol 2015.
14. Bailey S J, Winyard P, Vanhatalo A, et al. Dietary nitrate supplementation reduces the O2 cost of low-intensity exercise and enhances tolerance to high-intensity exercise in humans. J Appl Physiol (1985) 2009; 107:1144-55.
15. Honavar J, Doran S, Oh J Y, Steele C, Matalon S, Patel R P. Nitrite therapy improves survival postexposure to chlorine gas. Am J Physiol Lung Cell Mol Physiol 2014; 307:L888-94.
16. Samal A A, Honavar J, Brandon A, et al. Administration of nitrite after chlorine gas exposure prevents lung injury: effect of administration modality. Free Radic Biol Med 2012; 53:1431-9.
17. Yadav A K, Doran S F, Samal A A, et al. Mitigation of chlorine gas lung injury in rats by postexposure administration of sodium nitrite. Am J Physiol Lung Cell Mol Physiol 2011; 300:L362-9.
18. Ahmed K A, Nichols A L, Honavar J, Dransfield M T, Matalon S, Patel R P. Measuring nitrate reductase activity from human and rodent tongues. Nitric Oxide 2017; 66:62-70.
19. Aggarwal S, Jilling T, Doran S, et al. Phosgene inhalation causes hemolysis and acute lung injury. Toxicol Lett 2019; 312:204-13.
20. Honavar J, Bradley E, Bradley K, et al. Chlorine gas exposure disrupts nitric oxide homeostasis in the pulmonary vasculature. Toxicology 2014; 321:96-102.
21. Aggarwal S, Lam A, Bolisetty S, et al. Heme Attenuation Ameliorates Irritant Gas Inhalation-Induced Acute Lung Injury. Antioxid Redox Signal 2016; 24:99-112.
22. Sciuto A M, Moran T S, Narula A, Forster J S. Disruption of gas exchange in mice after exposure to the chemical threat agent phosgene. Mil Med 2001; 166:809-14.
23. Duniho S M, Martin J, Forster J S, et al. Acute changes in lung histopathology and bronchoalveolar lavage parameters in mice exposed to the choking agent gas phosgene. Toxicol Pathol 2002; 30:339-49.
24. Holmes W W, Keyser B M, Paradiso D C, et al. Conceptual approaches for treatment of phosgene inhalation-induced lung injury. Toxicol Lett 2016; 244:8-20.
25. Ahmad S, Ahmad A, Hendry-Hofer T B, et al. Sarcoendoplasmic reticulum Ca(2+) ATPase.
A critical target in chlorine inhalation-induced cardiotoxicity. Am J Respir Cell Mol Biol 2015; 52:492-502.
26. Honavar J, Samal A A, Bradley K M, et al. Chlorine gas exposure causes systemic endothelial dysfunction by inhibiting endothelial nitric oxide synthase-dependent signaling. Am J Respir Cell Mol Biol 2011; 45:419-25.
27. Zaky A, Bradley W E, Lazrak A, et al. Chlorine inhalation-induced myocardial depression and failure. Physiol Rep 2015; 3.
28. Ignarro L J, Napoli C. Novel features of nitric oxide, endothelial nitric oxide synthase, and atherosclerosis. Curr Atheroscler Rep 2004; 6:281-7.
29. Galkin A, Higgs A, Moncada S. Nitric oxide and hypoxia. Essays Biochem 2007; 43:29-42.
30. Duranski M R, Greer J J, Dej am A, et al. Cytoprotective effects of nitrite during in vivo ischemia-reperfusion of the heart and liver. J Clin Invest 2005; 115:1232-40.
31. Kelpke S S, Chen B, Bradley K M, et al. Sodium nitrite protects against kidney injury induced by brain death and improves post-transplant function. Kidney Int 2012; 82:304-13.
32. Kevil C G, Patel R P. Preserving vessel function during ischemic disease: new possibilities of inorganic nitrite therapy. Expert Rev Cardiovasc Ther 2008; 6:1175-9.
33. Vitturi D A, Patel R P. Current perspectives and challenges in understanding the role of nitrite as an integral player in nitric oxide biology and therapy. Free Radic Biol Med 2011; 51:805-12.
34. Gladwin M T, Raat N J, Shiva S, et al. Nitrite as a vascular endocrine nitric oxide reservoir that contributes to hypoxic signaling, cytoprotection, and vasodilation. Am J Physiol Heart Circ Physiol 2006; 291:H2026-35.
35. Omar S A, Webb A J, Lundberg J O, Weitzberg E. Therapeutic effects of inorganic nitrate and nitrite in cardiovascular and metabolic diseases. J Intern Med 2015.
36. Lundberg J O, Weitzberg E, Gladwin M T. The nitrate-nitrite-nitric oxide pathway in physiology and therapeutics. Nat Rev Drug Discov 2008; 7:156-67.
37. Bryan N S, Ivy J L. Inorganic nitrite and nitrate: evidence to support consideration as dietary nutrients. Nutr Res 2015; 35:643-54.
38. Tang Y, Jiang H, Bryan N S. Nitrite and nitrate: cardiovascular risk-benefit and metabolic effect. Curr Opin Lipidol 2011; 22:11-5.
39. Doel J J, Benjamin N, Hector M P, Rogers M, Allaker R P. Evaluation of bacterial nitrate reduction in the human oral cavity. Eur J Oral Sci 2005; 113:14-9.
40. Doel J J, Hector M P, Amirtham C V, Al-Anzan L A, Benjamin N, Allaker R P. Protective effect of salivary nitrate and microbial nitrate reductase activity against caries. Eur J Oral Sci 2004; 112:424-8.
41. Hyde E R, Andrade F, Vaksman Z, et al. Metagenomic analysis of nitrate-reducing bacteria in the oral cavity: implications for nitric oxide homeostasis. PLoS One 2014; 9:e88645.
42. Hyde E R, Luk B, Cron S, et al. Characterization of the rat oral microbiome and the effects of dietary nitrate. Free Radic Biol Med 2014; 77:249-57.
43. Duncan C, Li H, Dykhuizen R, et al. Protection against oral and gastrointestinal diseases: importance of dietary nitrate intake, oral nitrate reduction and enterosalivary nitrate circulation. Comp Biochem Physiol A Physiol 1997; 118:939-48.
44. Li H, Duncan C, Townend J, et al. Nitrate-reducing bacteria on rat tongues. Appl Environ Microbiol 1997; 63:924-30.
45. Jones J A, Hopper A O, Power G G, Blood A B. Dietary intake and bio-activation of nitrite and nitrate in newborn infants. Pediatr Res 2015; 77:173-81.
46. Hendgen-Cotta U B, Luedike P, Totzeck M, et al. Dietary nitrate supplementation improves revascularization in chronic ischemia. Circulation 2012; 126:1983-92.
47. Lundberg J O, Weitzberg E, Cole J A, Benjamin N. Nitrate, bacteria and human health. Nat Rev Microbiol 2004; 2:593-602.
48. Rathod K S, Velmurugan S, Ahluwalia A. A ‘green’ diet-based approach to cardiovascular health? Is inorganic nitrate the answer? Mol Nutr Food Res 2015.
49. Sciuto A M, Stotts R R, Chittenden V, Choung E, Heflin M D. Changes in absorbance at 413 nm in plasma from three rodent species exposed to phosgene. Biochem Biophys Res Commun 1996; 226:906-11.
50. Aggarwal S, Ahmad I, Lam A, et al. Heme scavenging reduces pulmonary endoplasmic reticulum stress, fibrosis, and emphysema. JCI Insight 2018; 3.
51. Lo S H, Chan C C, Chen W C, Wang J D. Grand rounds: outbreak of hematologic abnormalities in a community of people exposed to leakage of fire extinguisher gas. Environmental health perspectives 2006; 114:1713-7.
52. Baliga R S, Milsom A B, Ghosh S M, et al. Dietary nitrate ameliorates pulmonary hypertension: cytoprotective role for endothelial nitric oxide synthase and xanthine oxidoreductase. Circulation 2012; 125:2922-32.
53. Raat N J, Noguchi A C, Liu V B, et al. Dietary nitrate and nitrite modulate blood and organ nitrite and the cellular ischemic stress response. Free Radic Biol Med 2009; 47:510-7.
54. Delmastro-Greenwood M, Hughan K S, Vitturi D A, et al. Nitrite and nitrate-dependent generation of anti-inflammatory fatty acid nitroalkenes. Free Radic Biol Med 2015; 89:333-41.
55. Ferguson S K, Hirai D M, Copp S W, et al. Impact of dietary nitrate supplementation via beetroot juice on exercising muscle vascular control in rats. J Physiol 2013; 591:547-57.
56. Carlstrom M, Larsen F J, Nystrom T, et al. Dietary inorganic nitrate reverses features of metabolic syndrome in endothelial nitric oxide synthase-deficient mice. Proc Natl Acad Sci USA 2010; 107:17716-20.
57. Khambata R S, Ghosh S M, Rathod K S, et al. Antiinflammatory actions of inorganic nitrate stabilize the atherosclerotic plaque. Proc Natl Acad Sci USA 2017; 114:E550-E9.
58. Bondonno C P, Liu A H, Croft K D, et al. Antibacterial mouthwash blunts oral nitrate reduction and increases blood pressure in treated hypertensive men and women. Am J Hypertens 2015; 28:572-5.
59. Kautza B, Gomez H, Escobar D, et al. Inhaled, nebulized sodium nitrite protects in murine and porcine experimental models of hemorrhagic shock and resuscitation by limiting mitochondrial injury. Nitric Oxide 2015; 51:7-18.
60. Gladwin M T, Raat N J, Shiva S, et al. Nitrite as a vascular endocrine nitric oxide reservoir that contributes to hypoxic signaling, cytoprotection, and vasodilation. Am J Physiol Heart Circ Physiol 2006; 291:H2026-35.
61. Kevil C G, Kolluru G K, Pattillo C B, Giordano T. Inorganic nitrite therapy: historical perspective and future directions. Free radical biology & medicine 2011; 51:576-93.
62. Lundberg J O, Gladwin M T, Ahluwalia A, et al. Nitrate and nitrite in biology, nutrition and therapeutics. Nat Chem Biol 2009; 5:865-9.
63. Vitturi D A, Patel R P. Current perspectives and challenges in understanding the role of nitrite as an integral player in nitric oxide biology and therapy. Free Radic Biol Med 2011; 51:805-12.
64. Castiglione N, Rinaldo S, Giardina G, Stelitano V, Cutruzzola F. Nitrite and nitrite reductases: from molecular mechanisms to significance in human health and disease. Antioxidants & redox signaling 2012; 17:684-716.
65. Cosby K, Partovi K S, Crawford J H, et al. Nitrite reduction to nitric oxide by deoxyhemoglobin vasodilates the human circulation. Nat Med 2003; 9:1498-505.
66. Crawford J H, Isbell T S, Huang Z, et al. Hypoxia, red blood cells, and nitrite regulate NO-dependent hypoxic vasodilation. Blood 2006; 107:566-74.
67. Curtis E, Hsu L L, Noguchi A C, Geary L, Shiva S. Oxygen regulates tissue nitrite metabolism. Antioxidants & redox signaling 2012; 17:951-61.
68. Bryan N S. Nitrite in nitric oxide biology: cause or consequence? A systems-based review. Free radical biology & medicine 2006; 41:691-701.
69. Bryan N S, Fernandez B O, Bauer S M, et al. Nitrite is a signaling molecule and regulator of gene expression in mammalian tissues. Nat Chem Biol 2005; 1:290-7.
70. Feelisch M, Fernandez B O, Bryan N S, et al. Tissue processing of nitrite in hypoxia: an intricate interplay of nitric oxide-generating and -scavenging systems. J Biol Chem 2008; 283:33927-34.
71. Nagababu E, Ramasamy S, Abernethy D R, Rifkind J M. Active nitric oxide produced in the red cell under hypoxic conditions by deoxyhemoglobin-mediated nitrite reduction. J Biol Chem 2003; 278:46349-56.
72. Nagababu E, Ramasamy S, Rifkind J M. S-nitrosohemoglobin: a mechanism for its formation in conjunction with nitrite reduction by deoxyhemoglobin. Nitric oxide: biology and chemistry/official journal of the Nitric Oxide Society 2006; 15:20-9.
73. Roche C J, Dantsker D, Samuni U, Friedman J M. Nitrite reductase activity of sol-gel-encapsulated deoxyhemoglobin. Influence of quaternary and tertiary structure. J Biol Chem 2006; 281:36874-82.
74. van Faassen E E, Bahrami S, Feelisch M, et al. Nitrite as regulator of hypoxic signaling in mammalian physiology. Med Res Rev 2009; 29:683-741.
75. Zweier J L, Li H, Samouilov A, Liu X. Mechanisms of nitrite reduction to nitric oxide in the heart and vessel wall. Nitric oxide: biology and chemistry/official journal of the Nitric Oxide Society 2010; 22:83-90.
76. Dejam A, Hunter C J, Tremonti C, et al. Nitrite infusion in humans and nonhuman primates: endocrine effects, pharmacokinetics, and tolerance formation. Circulation 2007; 116:1821-31.
77. Pluta R M. Prolonged Intravenous Infusion of Sodium Nitrite Delivers Nitric Oxide (NO) in Humans. Acta Neurochir Suppl 2013; 115:49-51.
78. Pluta R M, Oldfield E H, Bakhtian K D, et al. Safety and feasibility of long-term intravenous sodium nitrite infusion in healthy volunteers. PloS one 2011; 6:e14504.
79. Bahra M, Kapil V, Pearl V, Ghosh S, Ahluwalia A. Inorganic nitrate ingestion improves vascular compliance but does not alter flow-mediated dilatation in healthy volunteers. Nitric Oxide 2012; 26:197-202.
80. Ingram T E, Pinder A G, Bailey D M, Fraser A G, James P E. Low-dose sodium nitrite vasodilates hypoxic human pulmonary vasculature by a means that is not dependent on a simultaneous elevation in plasma nitrite. American journal of physiology Heart and circulatory physiology 2010; 298:H331-9.
81. Ahluwalia A, Gladwin M, Coleman G D, et al. Dietary Nitrate and the Epidemiology of
Cardiovascular Disease: Report From a National Heart, Lung, and Blood Institute Workshop. J Am Heart Assoc 2016; 5.
82. Mensinga T T, Speijers G J, Meulenbelt J. Health implications of exposure to environmental nitrogenous compounds. Toxicol Rev 2003; 22:41-51.
83. Centers for Disease Control and Prevention (CDC). National Center for Health Statistics
(NCHS). National Health and Nutrition Examination Survey Questionnaire (or Examination Protocol, or Laboratory Protocol). Hyattsville, Md.: U.S. Department of Health and Human Services, Centers for Disease Control and Prevention. 2011-2012.
84. Sales of the leading mouthwash/dental rinse brands in the United States in 2014 (in million U.S. dollars). 2015. at http://www.statista.com/statistics/195543/sales-of-leading-us-mouthwash-brands-in-2012-and-2013/.)
85. Woessner M, Smoliga J M, Tarzia B, Stabler T, Van Bruggen M, Allen J D. A stepwise reduction in plasma and salivary nitrite with increasing strengths of mouthwash following a dietary nitrate load. Nitric Oxide 2016; 54:1-7.
86. McDonagh S T, Wylie U, Winyard P G, Vanhatalo A, Jones A M. The Effects of Chronic Nitrate Supplementation and the Use of Strong and Weak Antibacterial Agents on Plasma Nitrite Concentration and Exercise Blood Pressure. International journal of sports medicine 2015; 36:1177-85.
87. Stapley R, Owusu B Y, Brandon A, et al. Erythrocyte storage increases rates of NO and nitrite scavenging: implications for transfusion-related toxicity. Biochem J 2012; 446:499-508.
88. Nakahira K, Haspel J A, Rathinam V A, et al. Autophagy proteins regulate innate immune responses by inhibiting the release of mitochondrial DNA mediated by the NALP3 inflammasome. Nature immunology 2011; 12:222-30.
89. Yousefi S, Gold J A, Andina N, et al. Catapult-like release of mitochondrial DNA by eosinophils contributes to antibacterial defense. Nat Med 2008; 14:949-53.
90. Higdon A N, Benavides G A, Chacko B K, et al. Hemin causes mitochondrial dysfunction in endothelial cells through promoting lipid peroxidation: the protective role of autophagy. Am J Physiol Heart Circ Physiol 2012; 302:H1394-409.
91. Baek J H, D'Agnillo F, Vallelian F, et al. Hemoglobin-driven pathophysiology is an in vivo consequence of the red blood cell storage lesion that can be attenuated in guinea pigs by haptoglobin therapy. The Journal of clinical investigation 2012; 122:1444-58.
92. Belcher J D, Chen C, Nguyen J, et al. Heme triggers TLR4 signaling leading to endothelial cell activation and vaso-occlusion in murine sickle cell disease. Blood 2014; 123:377-90.
93. Ghosh S, Adisa O A, Chappa P, et al. Extracellular hemin crisis triggers acute chest syndrome in sickle mice. The Journal of clinical investigation 2013.
94. Larsen R, Gozzelino R, Jeney V, et al. A central role for free heme in the pathogenesis of severe sepsis. Sci Transl Med 2010; 2:51ra71.
95. Schaer D J, Buehler P W, Alayash A I, Belcher J D, Vercellotti G M. Hemolysis and free hemoglobin revisited: exploring hemoglobin and hemin scavengers as a novel class of therapeutic proteins. Blood 2013; 121:1276-84.
96. Stapley R, Rodriguez C, Oh J Y, et al. Red blood cell washing, nitrite therapy, and antiheme therapies prevent stored red blood cell toxicity after trauma-hemorrhage. Free Radic Biol Med 2015; 85:207-18.
97. Baker P R, Schopfer F J, O'Donnell V B, Freeman B A. Convergence of nitric oxide and lipid signaling: anti-inflammatory nitro-fatty acids. Free Radic Biol Med 2009; 46:989-1003.
98. Hogg N, Kalyanaraman B. Nitric oxide and lipid peroxidation. Biochim Biophys Acta 1999; 1411:378-84.
99. Yazji I, Sodhi C P, Lee E K, et al. Endothelial T LR4 activation impairs intestinal microcirculatory perfusion in necrotizing enterocolitis via eNOS-NO-nitrite signaling. Proc Natl Acad Sci USA 2013; 110:9451-6.
100. Kanady J A, Aruni A W, Ninnis J R, et al. Nitrate reductase activity of bacteria in saliva of term and preterm infants. Nitric Oxide 2012; 27:193-200.
101. Lal C V, Travers C, Aghai Z H, et al. The Airway Microbiome at Birth. Sci Rep 2016; 6:31023.
102. Taft D H, Ambalavanan N, Schibler K R, et al. Intestinal microbiota of preterm infants differ over time and between hospitals. Microbiome 2014; 2:36.
103. Lang J D, Jr., Teng X, Chumley P, et al. Inhaled N O accelerates restoration of liver function in adults following orthotopic liver transplantation. J Clin Invest 2007; 117:2583-91.
104. Kumar R, Eipers P, Little R B, et al. Getting started with microbiome analysis: sample acquisition to bioinformatics. Curr Protoc Hum Genet 2014; 82:18 8 1-29.
105. Ford D A, Honavar J, Albert C J, et al. Formation of chlorinated lipids post-chlorine gas exposure. J Lipid Res 2016; 57:1529-40.
106. Duerr M A, Palladino E ND, Hartman C L, et al. Bromofatty aldehyde derived from bromine exposure and myeloperoxidase and eosinophil peroxidase modify GSH and protein. J Lipid Res 2018; 59:696-705.
107. Oh J YH, J.; Xu, X.; Genschmer, K.; Zhong, M.; Lebensburger, J.; Marques, M.b.; Kerby, J. D.; Pittet, J. F.; Gaggar, A.; Patel, R. P. Absorbance and redox based approaches for measuring free heme and free hemoglobin in biological matrices. Redox Biol 2016; In Press.
108. Bor-Kucukatay M, Wenby R B, Meiselman H J, Baskurt O K. Effects of nitric oxide on red blood cell deformability. Am J Physiol Heart Circ Physiol 2003; 284:H1577-84.
109. Svistunenko D A, Patel R P, Voloshchenko S V, Wilson M T. The globin-based free radical of ferryl hemoglobin is detected in normal human blood. J Biol Chem 1997; 272:7114-21.
110. Patel R P, Svistunenko D A, Darley-Usmar V M, Symons M C, Wilson M T. Redox cycling of human methaemoglobin by H₂O₂ yields persistent ferryl iron and protein based radicals. Free Radic Res 1996; 25:117-23.
111. Shaver C M, Upchurch C P, Janz D R, et al. Cell-free hemoglobin: a novel mediator of acute lung injury. Am J Physiol Lung Cell Mol Physiol 2016; 310:L532-41.
112. Samal A, Honovar J, Brandon A, et al. Post Chlorine gas exposure administration of nitrite prevents lung injury: effect of administration modality. Free Rad Biol Med 2012; In Press.
113. Yadav A K, Bracher A, Doran S F, et al. Mechanisms and modification of chlorine-induced lung injury in animals. Proc Am Thorac Soc 2010; 7:278-83.
114. Moore K P, Holt S G, Patel R P, et al. A causative role for redox cycling of myoglobin and its inhibition by alkalinization in the pathogenesis and treatment of rhabdomyolysis-induced renal failure. J Biol Chem 1998; 273:31731-7.
115. Oh J Y, Stapley R, Harper V, Marques M B, Patel R P. Predicting storage-dependent damage to red blood cells using nitrite oxidation kinetics, peroxiredoxin-2 oxidation, and hemoglobin and free heme measurements. Transfusion 2015; 55:2967-78.
116. Kumar D, Branch B G, Pattillo C B, et al. Chronic sodium nitrite therapy augments ischemia-induced angiogenesis and arteriogenesis. Proc Natl Acad Sci USA 2008; 105:7540-5.
117. Simmons J D, Freno D R, Muscat C A, et al. Mitochondrial DNA damage associated molecular patterns in ventilator-associated pneumonia: Prevention and reversal by intratracheal DNase I. J Trauma Acute Care Surg 2017; 82:120-5.
118. Tan Y B, Mulekar S, Gorodnya O, et al. Pharmacologic Protection of Mitochondrial DNA Integrity May Afford a New Strategy for Suppressing Lung Ischemia-Reperfusion Injury. Ann Am Thorac Soc 2017; 14:5210-S5.
119. Lundberg J O. Cardiovascular prevention by dietary nitrate and nitrite. Am J Physiol Heart Circ Physiol 2009; 296:H1221-3.
120. Shiva S, Sack M N, Greer J J, et al. Nitrite augments tolerance to ischemia/reperfusion injury via the modulation of mitochondrial electron transfer. J Exp Med 2007; 204:2089-102.
121. Larsen F J, Schiffer T A, Borniquel S, et al. Dietary inorganic nitrate improves mitochondrial efficiency in humans. Cell Metab 2011; 13:149-59.
122. Larsen F J, Weitzberg E, Lundberg J O, Ekblom B. Dietary nitrate reduces maximal oxygen consumption while maintaining work performance in maximal exercise. Free Radic Biol Med 2010; 48:342-7.
123. Greenway F L, Predmore B L, Flanagan D R, et al. Single-dose pharmacokinetics of different oral sodium nitrite formulations in diabetes patients. Diabetes Technol Ther 2012; 14:552-60.

In view of the many possible embodiments to which the principles of the disclosed subject matter may be applied, it should be recognized that the illustrated embodiments are only examples of the disclosure and should not be taken as limiting the scope of the disclosure. Rather, the scope of the disclosure is defined by the following claims. We therefore claim all that comes within the scope and spirit of these claims.

1. A method of treating a lung injury in a subject, wherein the lung injury is caused by inhalation of a chemical, inhalation of smoke, or SARS-CoV-2 infection, comprising administering to the subject a therapeutically effective amount of a nitrite salt, wherein the chemical is not chlorine, thereby treating the lung injury.

2. The method of claim 1, wherein the chemical comprises bromide, methyl bromide, mustard gas, nitrogen mustard, phosgene, phosgene oxime, diphosgene, phosphine, ammonia, bromine, methyl isocyanate, hydrogen chloride, osmium tetroxide, phosphorous, sulfuryl fluoride, lewisite, chloroacetophenone, chlorobenzylidenemalononitrile, chloropicrin, bromobenzylcyanide, dibenzoxazepine, or a combination or two or more thereof.

3. The method of claim 1, wherein the chemical comprises bromine or phosgene gas.

4. The method of claim 1, wherein the smoke is from a fire, exhaust fumes or an explosion.

5. The method of claim 1, wherein the subject has pneumonia resulting from SARS-CoV-2 infection.

6. The method of claim 1, wherein the nitrite salt comprises sodium nitrite, potassium nitrite or arginine nitrite.

7. The method of claim 1, wherein the therapeutically effective amount of nitrite salt is about 5 mg to about 600 mg.

8. The method of claim 1, wherein the nitrite salt is administered as a formulation comprising the nitrite salt and an anti-caking agent.

9. The method of claim 8, wherein the concentration of the anti-caking agent in the formulation is at least 20 parts per million.

10. The method of claim 8, wherein the anti-caking agent comprises sodium bicarbonate.

11. The method of claim 1, wherein the nitrite salt is administered by an intramuscular, intravenous, subcutaneous, oral or inhalation route.

12. The method of claim 1, further comprising selecting a subject with a lung injury prior to administering the nitrite salt.

13. A formulation comprising a nitrite salt and an anti-caking agent, wherein the anti-caking agent is present at a concentration of at least 20 parts per million.

14. The formulation of claim 13, wherein the nitrite salt is sodium nitrite, potassium nitrite or arginine nitrite.

15. The formulation of claim 13, wherein the anti-caking agent comprises sodium bicarbonate.

16. A method of treating a disease or condition in a subject, comprising administering to the subject a therapeutically effective amount of the formulation of claim 13, wherein the disease or condition is selected from a lung injury, pulmonary hypertension, heart failure, cardiogenic shock, hypertension, respiratory failure, a metabolic syndrome, diabetes, a lipid disorder, an endocrine disorder, a gastroenterological disorder, hypoperfusion, inflammation, cystic fibrosis and aging.

17. The method of claim 16, wherein the lung injury is caused by inhalation of a chemical, inhalation of smoke, or SARS-CoV-2 infection.

18. The method of claim 16, wherein;
the disease or condition comprises pulmonary hypertension and heart failure with a preserved ejection fraction (HFpEF);
the heart failure comprises right-sided heart failure, left-sided heart failure or congestive heart failure;
the hypertension comprises chronic hypertension, acute hypertension, urgency hypertension, emergency hypertension, prehypertension or a combination thereof;
the respiratory failure comprises acute respiratory distress syndrome, respiratory failure from trauma or mechanical injury, respiratory failure due to pneumonia, respiratory failure resulting from an infection disease, respiratory failure resulting from pulmonary edema, respiratory failure resulting from pulmonary embolism, respiratory failure due to infant respiratory distress syndrome, respiratory failure due to interstitial lung disease, or respiratory failure resulting from an autoimmune disease; or
the lipid disorder comprises hypercholesterolemia, hypertriglyceridemia, or both.

19-23. (canceled)

24. A method of enhancing cardiovascular performance in a subject, comprising administering to the subject a therapeutically effective amount of the formulation of claim 13.

25. A method of providing a nutritional supplement to a subject, comprising administering to the subject the formulation of claim 13.
